(19) United States
(12) Patent Application Publication
(10) Pub. No.: US 2006/0003342 A1
(43) Pub. Date: Jan. 5, 2006

(54) Fetal RNA in amniotic fluid to determine gene expression in the developing fetus

(76) Inventors: Diana W. Bianchi, Brookline, MA (US); Paige B. Larrabee, Portland, OR (US); Kirby L. Johnson, North Quincy, MA (US)

Correspondence Address: CHOATE, HALL & STEWART LLP, TWO INTERNATIONAL PLACE, BOSTON, MA, 02110, US

(21) Appl. No.: 11/036,815

(22) Filed: Jan. 14, 2005

Related U.S. Application Data
(60) Provisional application No. 60/536,571, filed on Jan. 15, 2004.

(51) Int. Cl. C12Q 1/68; C07H 21/02

(52) U.S. Cl. 435/6; 536/23.1

(57) ABSTRACT

The present invention provides improved methods of prenatal diagnosis, monitoring, screening and/or testing. The invention is based, at least in part, on the discovery that amniotic fluid is a rich source of cell-free fetal RNA. Methods of isolation and analysis of fetal RNA are described, that can lead to information about fetal gene expression that is not available by other techniques. The inventive systems allow for a more comprehensive determination of a living human fetus' health, growth and development and for the prenatal diagnosis of a variety of diseases and conditions.

### PRIORITY INFORMATION

This application claims priority to Provisional Patent Application No. 60/536,571, entitled “Fetal RNA in Amniotic Fluid to Determine Gene Expression in the Developing Fetus” and filed Jan. 15, 2004. The Provisional Patent Application is incorporated herein by reference in its entirety.

### GOVERNMENT INTERESTS

The work described herein was funded by the National Institutes of Health (Grant No. NIH HD42053). The United States government may have certain rights in the invention.

### BACKGROUND OF THE INVENTION

Genetic disorders and congenital abnormalities (also called birth defects) occur in about 3 to 5% of all live births (A. Robinson and M. G. Linden, “Clinical Genetic Handbook”, 1993, Blackwell Scientific Publications: Boston, Mass.). Combined, genetic disorders and congenital abnormalities have been estimated to account for up to 30% of pediatric hospital admissions (C. R. Scriver et al., Can. Med. Assoc. J., 1973, 108: 1111-1115; E. W. Ling et al., Am. J. Perinatal., 1991, 8: 164-169) and to be responsible for about half of all childhood deaths in industrialized countries (R. J. Berry et al., Public Health Report, 1987, 102: 171-181; R. A. Hoekelman and I. B. Pless, Pediatrics, 1998, 82: 582-595). In the US, birth defects are the leading cause of infant mortality (R. N. Anderson et al., Month. Stat. Rep., 1997, Vol. 45, No 11, Suppl. 2, p. 55). Furthermore, genetic disorders and congenital anomalies contribute substantially to long-term disability; they are associated with enormous medical-care costs (A. Czeizel et al., Mutat. Res., 1984, 128: 73-103; Centers of Disease Control, Morb. Mortal. Weekly Rep., 1989, 38: 264-267; S. Kaplan, J. Am. Coll. Cardiol., 1991, 18: 319-320; C. Cunniff et al., Clin. Genet., 1995, 48: 17-22) and create a heavy psychological and emotional burden on those afflicted and/or their families. For these and other reasons, prenatal diagnosis has long been recognized as an essential facet of the clinical management of pregnancy itself as well as a critical step toward the detection, prevention, and, eventually, treatment of genetic disorders.

Currently, the most accurate prenatal diagnosis is provided by analysis of the fetal karyotype. Fetal cells for use in these karyotyping methods are traditionally isolated from samples of amniotic fluid (obtained by amniocentesis), chorionic villi (obtained by chorionic villus sampling), or fetal blood (obtained by cordocentesis or percutaneous umbilical cord blood sampling). Such analyses can reveal the presence of numerical and/or structural chromosomal abnormalities. In addition to requiring tedious, time-consuming and labor intensive steps (B. Eiben et al., Am. J. Hum. Genet., 1990, 47: 656-663), these methods have limited sensitivity and their standard level of resolution does not allow detection of small or subtle chromosomal aberrations, leaving a wide number of diseases and conditions undetected.

In the past decade, the application of molecular biological techniques to conventional chromosome analysis has generated new clinical cytogenetics tools that have enhanced the spectrum of disorders that can be diagnosed prenatally. In addition to these new techniques, new sources of fetal genetic material have also been explored. These include intact fetal cells present in the maternal circulation, whose analysis has been shown to allow prenatal diagnosis of fetal chromosome aneuploidy (S. Elias et al., Lancet, 1992, 340: 1033; D. W. Bianchi et al., Hum. Genet., 1992, 90: 368-370; D. Gänshirt-Ahlert et al., Am. J. Reprod. Immunol., 1993, 30: 193-200; J. L. Simpson et al., J. Am. Med. Assoc., 1993, 270: 2357-2361; F. de la Cruz et al., Fetal Diagn. Ther., 1998, 13: 380; and D. W. Bianchi et al., Am. J. Hum. Genet., 1997, 61: 822-829), and fetal DNA sequences present in the serum and plasma of pregnant women, which have successfully been used for the determination of fetal gender, identification of fetal rhesus D status, diagnosis of problematic pregnancies and of various prenatal conditions (Y. M. D. Lo et al., Lancet, 1997, 350: 485-487; B. Pertl and D. W. Bianchi, Obstet. Gynecol., 2001, 98: 483-490; Y. M. D. Lo et al., Clin. Chem., 1999, 45: 1747-1751).

The development of new technologies and the discovery of new sources of fetal genetic material have led to significant improvements over conventional methods of prenatal diagnosis. However, existing strategies are limited in terms of the information they can provide. For example, they do not allow the investigation of human fetal development in vivo. In particular, the expression pattern of genes during fetal development, which is a valuable piece of information to gain a better understanding of the genetic mechanisms responsible for normal and abnormal development processes in utero, is not available through these methods. Currently, fetal monitoring is limited to very crude non-invasive parameters determined through measurement of maternal uterine size, detection of the fetus' heartbeat, and/or evaluation of fetal anatomy by sonographic examination. Although technological advances and information from the Human Genome Project have made possible the development of microarrays that allow simultaneous detection and quantitation of tens of thousands of gene transcripts, fetal gene expression analysis has only been performed through tissue examination of human abortuses or using animal models for genes and developmental pathways that are conserved across the animal kingdom. Consequently, almost nothing is known about genes active in human early development that was directly studied in humans.

Therefore, methodologies that allow prenatal gene expression monitoring, which could provide information about the well-being, disease state, and normal versus abnormal development of the living fetus, are highly desirable.

### SUMMARY OF THE INVENTION

The present invention provides a system for assessing gene expression in a living human fetus. This system can be used to define gene expression patterns that correlate with developmental events, and allows for the assessment of a fetus' health, growth, and development, and for prenatal diagnosis of a variety of diseases and conditions. No other technology is available for determining gene expression pattern in a living human fetus.

In general, the present invention involves isolating fetal RNA from a sample of amniotic fluid, and analyzing the fetal RNA obtained. In preferred embodiments, the analysis provides qualitative or quantitative information about fetal gene expression. In certain embodiments, fetal RNA is isolated at multiple time points during gestation. The present invention allows particular gene expression patterns, or elements of such patterns, to be correlated with developmental events, and further allows comparison of observed gene expression patterns or components with patterns or components for which such a correlation has been established.

In one aspect, the present invention provides fetal RNA isolated from a sample of amniotic fluid. Preferably, isolated fetal RNA is obtained by: treating a sample of amniotic fluid obtained from a pregnant woman, such that fetal RNA present in the sample of amniotic fluid is extracted, resulting in amniotic fluid fetal RNA.

In certain embodiments, amniotic fluid fetal RNA is extracted after removal of substantially all cell populations from the sample of amniotic fluid; and consists essentially of cell-free fetal RNA. In other embodiments, some cell populations are removed from the sample of amniotic fluid before the treating step, resulting in a remaining amniotic material. When extracted from a sample of remaining amniotic material, fetal RNA comprises cell-free fetal RNA as well as fetal RNA from the cells still present in the remaining material. In yet other embodiments, fetal RNA is extracted from fetal cells isolated from the sample of amniotic fluid; optionally, the isolated cells are cultured before RNA extraction. In such cases, amniotic fluid fetal RNA consists essentially of fetal RNA from the cultured cells.

Preferably, cell populations are removed within two hours of obtaining the sample of amniotic fluid; more preferably, cells are removed immediately after obtaining the sample of amniotic fluid.

In certain embodiments, RNase inhibitor is added to the sample of amniotic fluid or to the remaining amniotic material, the sample of amniotic fluid or of remaining amniotic material is then frozen and stored for a certain period of time under suitable storage conditions (for example, at −80° C.). Preferably, when the sample of amniotic fluid or of remaining amniotic material is to be frozen, the RNase inhibitor is added within two hours of obtaining the sample. More preferably, the RNase inhibitor is added immediately after obtaining the sample of amniotic fluid.

In certain embodiments, the amniotic fluid fetal RNA is amplified, for example, using one or more fetal sequence specific oligonucleotides, resulting in amplified fetal RNA. In certain embodiments, the amniotic fluid fetal RNA is messenger RNA (mRNA). In other embodiments, the amniotic fluid fetal RNA is converted into complementary DNA (cDNA) by reverse transcriptase, resulting in fetal cDNA. In still other embodiments, the amniotic fluid fetal RNA is converted into cDNA, which is, in turn, converted into complementary RNA (cRNA) by transcription, resulting in fetal cRNA.

In another aspect, the present invention provides methods of prenatal diagnosis, which comprise steps of: providing a sample of amniotic fluid fetal RNA; analyzing the amniotic fluid RNA to obtain information regarding the RNA; and based on the information obtained, providing a prenatal diagnosis. Preferably, the amniotic fluid fetal RNA is obtained by: treating a sample of amniotic fluid obtained from a pregnant woman such that fetal RNA present in the amniotic fluid is extracted and made available for analysis, resulting in amniotic fluid fetal RNA.

In certain embodiments, when the sample of amniotic fluid is to be processed (as opposed to frozen and stored), substantially all cell populations are removed from the sample of amniotic fluid and the extracted amniotic fluid fetal RNA consists essentially of cell-free fetal RNA. In other embodiments, some cell populations are removed from the amniotic fluid material to obtain a remaining amniotic material. In such cases, the remaining material still contains some cells and the extracted amniotic fluid fetal RNA comprises cell-free fetal RNA as well as RNA originating from the cells present in the remaining amniotic material. In still other embodiments, fetal RNA is extracted from cell populations isolated from the sample of amniotic fluid. Optionally, the isolated cells are cultured before RNA extraction. Preferably, cell populations are removed within two hours. More preferably, the cell populations are removed immediately after obtaining the sample of amniotic fluid.

In certain embodiments, an RNase inhibitor is added to the sample of amniotic fluid or of remaining amniotic material, the sample is then frozen and stored for a certain period of time under suitable storage conditions (for example, at −80° C.). At the time of analysis, the frozen sample is thawed and any remaining cell populations may be removed before treatment. Preferably, when the sample of amniotic fluid or of remaining amniotic material is to be frozen, the RNase inhibitor is added within two hours of obtaining the sample; more preferably, immediately after obtaining the amniotic fluid sample.

In certain embodiments of the inventive methods, the amniotic fluid fetal RNA is amplified before being analyzed, for example, using one or more fetal sequence specific oligonucleotides. In other embodiments, the amniotic fluid fetal RNA is mRNA. In still other embodiments, the amniotic fluid fetal RNA is converted into complementary DNA (cDNA) by reverse transcriptase prior to analysis. In yet other embodiments, prior to the analysis, the extracted amniotic fluid fetal RNA is converted into cDNA, which is, in turn, converted into complementary RNA (cRNA) by transcription.

In certain embodiments, the amniotic fluid fetal RNA (for example, after amplification or transcription) is labeled with a detectable agent prior to the analyzing step. The detectable agent may comprise a fluorescent label, a colorimetric label, a chemiluminescent label, a radionuclide, a magnetic label, a hapten, a microparticle, an enzyme, a detectable biological molecule and any combination thereof. Suitable fluorescent labels comprise fluorescent dyes such as Cy-3™, Cy-5™, Texas Red, FITC, phycoerythrin, rhodamine, fluorescein, fluorescein isothiocyanate, carbocyanine, merocyanine, styryl dye, oxonol dye, BODIPY dye, and equivalents, analogues, derivatives and combinations of these compounds. Suitable haptens include, for example, biotin and dioxigenin.

In certain embodiments of the inventive methods of prenatal diagnosis, the extracted amniotic fluid fetal RNA is fragmented before being analyzed.

In certain embodiments, analyzing the amniotic fluid fetal RNA comprises determining the quantity of fetal RNA. In other embodiments, analyzing the extracted amniotic fluid fetal RNA comprises determining the concentration of fetal RNA. In yet other embodiments, analyzing the amniotic fluid fetal RNA comprises determining the sequence composition of fetal RNA. In still other embodiments, analyzing the amniotic fluid fetal RNA comprises submitting the extracted fetal RNA to a gene analysis, for example, a gene expression analysis.

In certain embodiments, analyzing the amniotic fluid fetal RNA comprises using an array, such as a cDNA array, an oligonucleotide array, a SNP array or a gene expression array.

Analysis of the amniotic fluid fetal RNA may lead to information regarding the quantity, concentration, or sequence composition of fetal RNA, or to qualitative and/or quantitative information about gene expression. In the inventive methods, the information obtained by analysis of amniotic fluid fetal RNA is used to provide a prenatal diagnosis. In certain embodiments, providing a prenatal diagnosis comprises determining the sex of the fetus. In other embodiments, providing a prenatal diagnosis comprises assessing the developmental progress of the fetus. In still other embodiments, providing a prenatal diagnosis comprises identifying a disease or condition affecting the fetus.

The methods of prenatal diagnosis of the present invention may be performed when the fetus is suspected of having a disease or condition. The methods of the invention may be carried out on pregnant women of any age. In certain embodiments, the methods of the invention are carried out when the pregnant woman is 35 or more than 35 years old.

In another aspect, the present invention provides methods for establishing gene expression in a fetus. The inventive methods comprise steps of: providing a test sample of amniotic fluid fetal RNA, wherein the test sample comprises a plurality of nucleic acid segments labeled with a detectable agent; providing a gene-expression array comprising a plurality of genetic probes, wherein each genetic probe is immobilized to a discrete spot on a substrate surface to form the array; contacting the array with the test sample under conditions wherein the nucleic acid segments in the sample specifically hybridize to the genetic probes on the array; determining the binding of individual nucleic acid segments of the test sample to individual genetic probes immobilized on the array to obtain a binding pattern; and based on the binding pattern obtained, establishing a gene expression pattern for the fetus.

In certain embodiments, the methods of the invention further comprise: correlating one or more feature(s) of the gene expression pattern obtained with fetal gender and/or gestational age.

In certain embodiments, the fetus is karyotypically and developmentally normal. In other embodiments, the fetus is karyotypically abnormal. In still other embodiments, the fetus is developmentally abnormal. In yet other embodiments, the fetus is affected with a clinical condition.

In certain embodiments, the methods of the invention further comprise: repeating all the steps for a statistically significant number of amniotic fluid fetal RNA samples from karyotypically and developmentally normal male (or female) fetuses of different gestational ages; and based on the gene expression patterns obtained, establishing baseline levels of mRNA expression at different gestational ages in karyotypically and developmentally normal male (or female) fetuses.

In other embodiments, the methods of the invention further comprise: correlating one or more feature(s) of the gene expression pattern with a time or event in fetal development of a karyotypically and developmentally normal male fetus if the amniotic fluid fetal RNA analyzed is from a male fetus, or with a time or event in fetal development of a karyotypically and developmentally normal female fetus if the amniotic fluid fetal RNA analyzed is from a female fetus.

In still other embodiments, the methods of the invention further comprise: repeating all the steps for a statistically significant number of amniotic fluid fetal RNA samples from karyotypically and developmentally normal male (or female) fetuses of different gestational ages; correlating one or more feature of the gene expression patterns obtained with a time or event in fetal development of a karyotypically and developmentally normal male (or female) fetus; and based on the correlations, establishing a developmental gene expression pattern for karyotypically and developmentally normal male (or female) fetuses at different gestational ages.

In yet other embodiments, the methods of the invention further comprise: repeating all the steps for a statistically significant number of amniotic fluid fetal RNA samples from karyotypically abnormal fetuses with an identical chromosomal abnormality; comparing each gene expression pattern obtained with baseline levels of mRNA expression established for karyotypically and developmentally normal fetuses of similar gestational age and gender; and based on the comparison, identifying one or more gene(s) abnormally expressed in the karyotypically abnormal fetuses, and associated with the chromosomal abnormality. Preferably, the one or more gene(s) abnormally expressed is/are then catalogued as a function of chromosomal abnormality.

The chromosomal abnormality may be an extra individual chromosome, a missing individual chromosome, an extra portion of a chromosome, a missing portion of a chromosome, a break, a ring, an addition, a deletion, a translocation, an inversion, a duplication, and any combination of these. In certain embodiments, the chromosomal abnormality is associated with a disease or condition. The disease or condition may be an aneuploidy, such as, for example, Down syndrome, Patau syndrome, Edward syndrome, Turner syndrome, Klinefelter syndrome and XYY disease. The disease or condition associated with a chromosomal abnormality may be an X-linked disorder. Alternatively, the disease or condition associated with a chromosomal abnormality may be a microdeletion/microduplication syndrome.

In certain embodiments, the methods of the invention further comprise: repeating all the steps for a statistically significant number of amniotic fluid fetal RNA samples from developmentally abnormal fetuses with an identical developmental disease or condition; comparing each gene expression pattern obtained with baseline levels of mRNA expression established for karyotypically and developmentally normal fetuses of similar gestational age and gender; and based on the comparison, identifying one or more gene(s) abnormally expressed in the developmentally abnormal fetuses, and associated with the developmental disease or condition. Preferably, the one or more gene(s) abnormally expressed is/are then catalogued as a function of developmental disease or condition.

Developmental diseases or conditions affecting the fetus whose RNA can be analyzed by the methods of the invention may be intrauterine growth restriction, polyhydramnios, twin-to-twin transfusion (TTT) syndrome, pulmonary hypoplasia, oligohydramnios, infant of diabetic mother, club foot, amniotic bands, spina bifida, congenital diaphragmatic hernia, and renal dysplasia.

In other embodiments, the inventive methods further comprise steps of: repeating all the steps for a statistically significant number of amniotic fluid fetal RNA samples from diseased fetuses affected with an identical clinical condition; comparing each gene expression pattern obtained with baseline levels of mRNA expression established for karyotypically and developmentally normal fetuses of similar gestational age and gender; and based on the comparison, identifying one or more gene(s) abnormally expressed in the diseased fetuses, and associated with the clinical condition. Preferably, the one or more gene(s) abnormally expressed is/are then catalogued as a function of clinical condition.

The clinical condition affecting the diseased fetus may be viral, bacterial or parasitic infection, preterm labor, or preeclampsia.

As described above, the amniotic fluid fetal RNA used in the methods of the invention may consist essentially of cell-free fetal RNA; may further comprise fetal RNA from cells present in the sample of amniotic fluid or may consist essentially of fetal RNA from cultured cells isolated from a sample of amniotic fluid. As described above, the amniotic fluid fetal RNA may be extracted from a frozen sample of amniotic fluid or of remaining amniotic material.

The amniotic fluid fetal RNA to be used in the inventive methods for establishing gene expression pattern of a fetus, may be amplified, transcribed, labeled and/or fragmented as described above.

In certain embodiments, the hybridization capacity of high copy number repeat sequences present in the nucleic acids of the test sample is suppressed. For example, the hybridization capacity of the repetitive sequences is suppressed by adding to the test sample unlabeled blocking nucleic acids before the contacting step. Preferably, an excess of unlabeled blocking nucleic acids is added. In certain embodiments, the unlabeled blocking nucleic acids are Human Cot-1 DNA.

In certain embodiments, determining the binding of individual nucleic acid segments of the test sample to individual genetic probes immobilized on the array to obtain a binding pattern includes measuring the intensity of the signals produced by the detectable agent at each discrete spot on the array.

In other preferred embodiments, determining the binding of individual nucleic acid segments of the test sample to individual genetic probes immobilized on the array to obtain a binding pattern includes steps of: using a computer-assisted imaging system to obtain a fluorescence image of the array after hybridization; and using a computer-assisted image analysis system to analyze the fluorescence image obtained, to interpret data imaged from the array and to display results as fluorescence intensity as a function of genomic locus.

In another aspect, the present invention provides methods of prenatal diagnosis performed by submitting amniotic fluid fetal RNA to an array-based gene-expression analysis. The inventive methods comprise steps of: providing a test sample of amniotic fluid fetal RNA, wherein the fetal RNA comes from a sample of amniotic fluid obtained from a woman pregnant with a fetus of known gender and gestational age, and wherein the test sample comprises a plurality of nucleic acid segments labeled with a detectable agent; providing a gene-expression array comprising a plurality of genetic probes, wherein each genetic probe is immobilized to a discrete spot on a substrate surface to form the array; contacting the array with the test sample of amniotic fluid fetal RNA under conditions where the nucleic acid segments in the sample specifically hybridize to the genetic probes on the array; determining the binding of individual nucleic acid segments of the test sample to individual genetic probes immobilized on the array to obtain a binding pattern; based on the binding pattern obtained establishing a gene expression pattern for the fetus; analyzing the gene expression pattern; and based on the analysis of the gene expression pattern, providing a prenatal diagnosis.

In certain embodiments, analyzing the gene expression pattern comprises comparing the gene expression pattern of the fetus to baseline levels of mRNA expression established for karyotypically and developmentally normal fetuses of identical gender and gestational age. In other embodiments, analyzing the gene expression pattern comprises comparing the gene expression pattern of the fetus to a developmental gene expression pattern established for karyotypically and developmentally normal fetuses of identical gender and gestational age. In yet other embodiments, analyzing the gene expression pattern comprises detecting and/or identifying one or more gene(s) abnormally expressed. The one or more gene(s) abnormally expressed may be associated with a chromosomal abnormality, a developmental condition or another type of clinical condition or disease.

Providing a prenatal diagnosis according to the methods of the invention may comprise determining the developmental progress of the fetus and/or identifying a disease or condition affecting the fetus.

The inventive methods may be carried out on pregnant women of any age. In certain embodiments, the inventive methods are carried out when the pregnant woman is 35 or more than 35 years old. In other embodiments, the inventive methods are carried out when the fetus is suspected of having a disease or condition, for example, a disease or condition associated with a chromosomal abnormality, a developmental anomaly or another clinical disorder.

In certain embodiments, the gender of the fetus is determined by analysis of the karyotype of the fetus established by G-banding analysis, metaphase comparative genomic hybridization, fluorescence in situ hybridization or spectral karyotyping. In other embodiments, the gestational age has been determined by sonographic examination.

In the practice of the inventive methods, amniotic fluid fetal RNA may be isolated, amplified, transcribed, labeled, fragmented, stored and/or hybridized as described above. Similarly detection of the binding of nucleic acid segments of the test sample of amniotic fluid fetal RNA to genetic probes immobilized on the array may be determined as described above.

In another aspect, the present invention provides kits containing some or all of the following components: materials to extract cell-free fetal RNA from a sample of amniotic fluid obtained from a pregnant woman; a gene-expression array comprising a plurality of genetic probes, wherein each genetic probe is immobilized to a discrete spot on a substrate surface to form the array; a database comprising baseline levels of mRNA expression established for karyotypically and developmentally normal male, and normal female fetuses at different gestational ages; a database comprising developmental gene expression patterns established for karyotypically and developmentally normal male, and normal female fetuses at different gestational ages, wherein one or more feature(s) of the developmental gene expression pattern is/are correlated with a time or event in fetal development; and instructions for using the extraction materials, array and databases according to the methods of the invention.

The inventive kits may also contain materials to label samples of nucleic acids with a detectable agent, for example, with a fluorescent dye, such as, Cy-3™, Cy-5™, Texas Red, FITC, phycoerythrin, rhodamine, fluorescein, fluorescein isothiocyanate, carbocyanine, merocyanine, styryl dye, oxonol dye, BODIPY dye, equivalents, analogues, derivatives, and combinations of these compounds, or with a hapten, such as, for example, a biotin/avidin system.

The inventive kits may also comprise, in individual containers, hybridization and wash buffers, RNase inhibitor, carrier RNA and/or Human Cot-1 DNA.

Other aspects, features and advantages of the present invention will become apparent from the following detailed description. It should be understood, however, that the detailed description and specific examples, while indicating preferred embodiments of the invention, are given by way of illustration only.

### BRIEF DESCRIPTION OF THE DRAWING

FIG. 1 is a picture of an agarose gel (1% agarose/ethidium bromide stained), showing the samples of fragmented cell-free amniotic fluid RNA (sample TTT1 in lane 2; and sample Hydrops1 in lane 3) compared to a molecular weight marker (in lane 1).

FIG. 2 is a table summarizing the levels of mRNA expression from sample Hydrops1 (male) compared to those from sample TTT1 (female).

FIG. 3 is a picture of an agarose gel (1% agarose/ethidium bromide stained) showing in lanes 2 and 3, sample TTT3; in lanes 4 and 5, sample TTT2; in lanes 6 and 7, sample Hydrops2; in lanes 8 and 9, pooled male control sample; in lanes 10 and 11, pooled female control sample, before and after fragmentation, respectively. Lanes 1 and 12 were loaded with a molecular weight marker, for comparison.

FIG. 4 is a table listing the genes that were found to exhibit the most statistically significant different (>4 fold difference) levels of expression in TTT fetuses (TTT1 and TTT2) compared to the 17-week male control.

FIG. 5 is a table listing the genes that were found to exhibit the most statistically significant different (>4 fold difference) levels of expression in hydrops fetuses (Hydrops1 and Hydrops2) compared to the 17-week male control.

### DEFINITIONS

Unless otherwise stated, all technical and scientific terms used herein have the meaning commonly understood by a person skilled in the art to which this invention belongs. The following terms have the meaning ascribed to them unless specified otherwise.

As used herein, the term “prenatal diagnosis” refers to the determination of the health and conditions of a fetus, including the detection of defects or abnormalities as well as the diagnosis of diseases. A variety of non-invasive and invasive techniques are available for prenatal diagnosis. Each of them can be used only during specific time periods of the pregnancy for greatest utility. These techniques include, for example, ultrasonography, maternal serum screening, amniocentesis, and chorionic villus sampling (or CVS). The inventive methods of prenatal diagnosis include analysis of fetal RNA isolated from amniotic fluid.

The terms “sonographic examination”, “ultrasonographic examination”, and “ultrasound examination” are used herein interchangeably. They refer to a clinical non-invasive procedure in which high frequency sound waves are used to produce visible images from the pattern of echos made by different tissues and organs of the fetus. A sonographic examination may be used to determine the size and/or position of the fetus, the size and/or position of the placenta, the amount of amniotic fluid, the appearance of fetal anatomy, and/or the fetus' age (or gestational age). Ultrasound examinations can also reveal the presence of congenital anomalies (i.e., anatomical or structural malformations that are present at birth), or of developmental abnormalities.

The term “amniocentesis”, as used herein, refers to a prenatal test performed by inserting a long needle through the mother's lower abdomen and into the amniotic cavity inside the uterus, using ultrasound to guide the needle, and withdrawing a small amount of amniotic fluid if the amniocentesis is performed for diagnostic purposes or a larger amount of amniotic fluid if the amniocentesis is performed for therapeutic purposes. The amniotic fluid may contain skin, kidney, and lung cells from the fetus. In conventional amniocentesis, these cells are grown in culture and tested for chromosomal abnormalities by determination and analysis of their karyotypes and the amniotic fluid itself can be tested for biochemical abnormalities. As discovered by the Applicants (see below), the amniotic fluid also contains cell-free fetal nucleic acids.

The term “nucleic acid” and “nucleic acid molecule” are used herein interchangeably. They refer to a deoxyribonucleotide or ribonucleotide polymer in either single-or double-stranded form, and unless otherwise stated, encompass known analogs of natural nucleotides that can function in a similar manner as naturally occurring nucleotides. The terms encompass nucleic acid-like structures with synthetic backbones, as well as amplification products.

The term “isolated” when applied to fetal RNA means a molecule of RNA or a portion thereof, which by virtue of its origin or manipulation, is separated from at least some of the components with which it is naturally associated. By “isolated”, it is alternatively or additionally meant that the RNA molecule of interest is produced or synthesized by the hand of man.

The term “made available for analysis” is used herein to specify that amniotic fluid fetal RNA is manipulated (e.g., amplified, transcribed, labeled, fragmented, purified, and/or concentrated and resuspended in a soluble aqueous solution) such that it is in a form suitable for analysis.

As used herein, the term “amnioticfluidfetal RNA” refers to a RNA molecule of fetal origin that has a sequence identical or complementary to that of RNA found in a sample of amniotic fluid. The term encompasses fetal total RNA, mRNA, cDNA and cRNA derived from RNA extracted from amniotic fluid.

The term “messenger RNA or mRNA” refers to a form of RNA that serves as a template to direct protein biosynthesis. Typically, the amount of any particular type of mRNA (i.e., having the same sequence, and originating from the same gene) reflects the extent to which a gene has been “expressed”.

The term “gene expression” refers to the process by which RNA and proteins are made from the instructions encoded in genes. Alterations in gene expression can change the function of the cell, tissue, organ, or whole organism and sometimes result in observable characteristics associated with a particular gene. Gene expression monitoring may be used to examine individual genes, groups of related genes, interlocking biochemical pathways, and biological networks as a whole.

As used herein, the term “gene” refers to a part of the genome specifying a macromolecular product, be it RNA or a protein, and may include regulatory sequences preceding (5′ non-coding sequences) and following (3′ non-coding sequences) the coding sequence.

The term “RNA transcript” refers to the product resulting from transcription of a DNA sequence. When the RNA transcript is the original, unmodified product of a RNA polymerase catalyzed transcription, it is referred to as the primary transcript. An RNA transcript that has been processed (e.g., spliced, etc) will differ in sequence from the primary transcript; a fully processed transcript is referred to as a “mature” RNA. The term “transcription” refers to the process of copying a DNA sequence of a gene into a RNA product, generally conducted by a DNA-directed RNA polymerase using the DNA as a template. A processed RNA transcript that is translated into protein is often called a messenger RNA (mRNA).

The term “complementary DNA or cDNA” refers to a DNA molecule that is complementary to mRNA. cDNA can be made by DNA polymerase (e.g., reverse transcriptase) or by directed chemical synthesis. The term “complementary” refers to nucleic acid sequences that base-pair according to the standard Watson-Crick complementary rules, or that is capable of hybridizing to a particular nucleic acid segment under relatively stringent conditions. Nucleic acid polymers are optionally complementary across only portions of their entire sequences.

The terms “array”, “micro-array”, and “biochip” are used herein interchangeably. They refer to an arrangement, on a substrate surface, of multiple nucleic acid molecules of known sequences. Each nucleic acid molecule is immobilized to a “discrete spot” (i.e., a defined location or assigned position) on the substrate surface. The term “micro-array” more specifically refers to an array that is miniaturized so as to require microscopic examination for visual evaluation. Arrays used in the methods of the invention are preferably microarrays.

The term “gene expression array” refers to an array comprising a plurality of genetic probes immobilized on a substrate surface that can be used for quantitation of mRNA expression levels. In the context of the present invention, the term “array-based gene expression analysis” is used to refer to methods of gene expression analysis that use gene-expression arrays. The term “genetic probe”, as used herein, refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acid fragments from a test sample of amniotic fluid fetal RNA are hybridized. Genetic probes specifically bind (or specifically hybridize) to nucleic acid of complementary or substantially complementary sequence through one or more types of chemical bonds, usually through hydrogen bond formation.

The term “oligonucleotide”, as used herein, refers to usually short strings of DNA or RNA to be used as hybridizing probes or nucleic acid molecule array elements. These short stretches of sequence are often chemically synthesized. The size of the oligonucleotide depends on the function or use of the oligonucleotides. When used in microarrays for hybridization, oligonucleotides can comprise natural nucleic acid molecules or synthesized nucleic acid molecules and comprise between about 5 and about 150 nucleotides, preferably between about 15 and about 100 nucleotides, more preferably between about 15 and about 30 nucleotides and most preferably, between about 18 and about 25 nucleotides complementary to mRNA.

The terms “genetic site”, “genetic locus” and “genomic locus” are used herein interchangeably. They refer to a specific region of the genome. In the methods of the invention, each genetic probe immobilized to a discrete spot on an array has a sequence that is specific to (or characteristic of) a particular genomic locus.

The term “hybridization” refers to the binding of two single stranded nucleic acids via complementary base pairing. The terms “specific hybridization” (or “specifically hybridizes to”) and “specific binding” (or “specifically binds to”) are used herein interchangeably. They refer to a process in which a nucleic acid molecule preferentially binds, duplexes, or hybridizes to a particular nucleic acid sequence under stringent conditions (e.g., in the presence of competitor nucleic acids with a lower degree of complementarity to the hybridizing strand). In certain embodiments of the present invention, these terms more specifically refer to a process in which a nucleic acid fragment (or segment) from a test sample preferentially binds to a particular genetic probe immobilized on an array and to a lesser extent, or not at all, to other arrayed genetic probes.

The terms “gene expression pattern” and “gene expression profile” are used herein interchangeably. They refer to the expression of an individual gene or of suites of individual genes. A gene expression pattern may include information regarding the presence of target transcripts in a sample, and the relative or absolute abundance levels of target transcripts. Additionally or alternatively, gene expression pattern may include information regarding the ability of a prenatal treatment to induce expression of specific genes or the ability of a prenatal treatment to change the expression of specific genes to different levels.

The term “karyotypically and developmentally normal fetus” is used herein to designate a fetus whose karyotype is determined to be normal (i.e., a karyotype that does not contain chromosomal abnormalities) and whose development has been determined to be appropriate for gestational age, for example, by sonographic examination.

As used herein, the term “karyotype” refers to the particular chromosome complement of an individual, as defined by the number and morphology of the chromosomes usually in mitotic metaphase. More specifically, a karyotype includes such information as total chromosome number, copy number of individual chromosome types (e.g., the number of copies of chromosome Y) and chromosomal morphology (e.g., length, centromeric index, connectedness and the like). Examination of a karyotype allows detection and identification of chromosomal abnormalities (e.g., extra, missing, or broken chromosomes). Since certain diseases and conditions are associated with characteristic chromosomal abnormalities, analysis of a karyotype usually allows diagnosis of some diseases and conditions.

The term “chromosome” has herein its art understood meaning. It refers to structures composed of very long DNA molecules (and associated proteins) that carry most of the hereditary information of an organism. Chromosomes are divided into functional units called “genes”, each of which contains the genetic code (i.e., instructions) for making a specific protein or RNA molecule. In humans, a normal body cell contains 46 chromosomes; a normal reproductive cell contains 23 chromosomes.

The terms “chromosomal abnormality”, “chromosomal aberration” and “chromosomal alteration” are used herein interchangeably. They refer to a difference (i.e., a variation) in the number of chromosomes or to a difference (i.e., a modification) in the structural organization of one or more chromosomes as compared to chromosomal number and structural organization in a karyotypically normal individual. As used herein, these terms are also meant to encompass abnormalities taking place at the gene level. The presence of an abnormal number of (i.e., either too many or too few) chromosomes is called “aneuploidy”. Examples of aneuploidy are trisomy 21 and trisomy 13. Structural chromosomal abnormalities include: deletions (e.g., absence of one or more nucleotides normally present in a gene sequence, absence of an entire gene, or missing portion of a chromosome), additions (e.g., presence of one or more nucleotides usually absent in a gene sequence, presence of extra copies of a gene (also called duplication), or presence of an extra portion of a chromosome), rings, breaks and chromosomal rearrangements. Chromosomal rearrangements may be translocations or inversions. A translocation results from a process in which genetic material is transferred from one gene to another. A translocation is balanced when two chromosomes exchange pieces without loss of genetic material, while an unbalanced translocation occurs when chromosomes either gain or lose genetic material. Translocations may involve two chromosomes or only one chromosome. Inversions are produced by a process in which two breaks occur in a chromosome and the broken segment rotates 180°, resulting in the genes being rearranged in reverse order.

As used herein, the terms “microdeletion”, and “microduplication”, refer to subtle, cryptic or small chromosomal abnormalities (for example involving one or more nucleotides in a gene sequence, or involving loss or gain of a single gene copy) that cannot be detected or are not easily detectable by standard cytogenetic methods, such as, for example, conventional G-banding analysis or metaphase comparative genomic hybridization.

As used herein, the term “disease or condition associated with a chromosomal abnormality” refers to any disease, disorder, condition or defect, that is known or suspected to be caused by a chromosomal abnormality. Exemplary diseases or conditions associated with a chromosomal abnormality include, but are not limited to, trisomies (e.g., Down syndrome, Edward syndrome, Patau syndrome, Turner syndrome, Klinefelter syndrome, and XYY disease), X-linked disorders (e.g., Duchenne muscular dystrophy, hemophilia A, certain forms of severe combined immunodeficiency, Lesch-Nyhan syndrome, and Fragile X syndrome) and microdeletion/microduplication syndromes (e.g., Prader-Willi syndrome, Angelman syndrome, DiGeorge syndrome, Smith-Magenis syndrome, Rubinstein-Taybi syndrome, Miller-Dieker syndrome, Williams syndrome, and Charcot-Marie-Tooth syndrome). Additional examples of diseases or conditions associated with chromosomal abnormalities may be found in “Harrison's Principles of Internal Medicine”, Wilson et al. (Eds.), 1991 (12^{th} Ed.), Mc Graw Hill: New York, N.Y., pp 24-46.

As used herein, the term “G-banding or Giemsa banding” refers to a standard staining technique for karyotyping. G-banding (also known as G-T-G banding) involves the use of an enzyme (the protease trypsin) to degrade some of the proteins that are associated with the chromosomes and the use of a staining dye (Giemsa) that selectively binds to DNA regions rich in guanine and cytosine. This selective staining leads to the creation of a distinctive pattern of alternating dark and light bands along the length of the chromosome, that is characteristic of the individual chromosome (light bands correspond to euchromatin, which is active DNA rich in guanine and cytosine; dark bands are heterochromatin, which is unexpressed DNA rich in adenine and thymine). This staining reveals extra and missing chromosomes, large deletions and duplications, as well as the locations of centromeres (the major constrictions in chromosomes). However less extensive or more complex rearrangements of genetic material, chromosomal origins of markers, and subtle translocations are not detectable or are difficult to identify with certainty using standard G-banding (Giemsa, Leishman's or variant). For more details on how to perform a G-banding analysis, see, for example, J. M. Scheres et al., Hum. Genet., 1982, 61: 8-11; and K. Wakui et al., J. Hum. Genet., 1999, 44: 85-90.

As used herein, the term “Fluorescence In Situ Hybridization or FISH” refers to a molecular cytogenetic technique that can be used to generate karyotypes. In a FISH experiment, specifically designed fluorescent molecules are used to visualize particular genes or sections of chromosomes by fluorescence microscopy, thus allowing detection of chromosomal abnormalities (see, for example, T. Bryndorf et al., Acta Obstet. Gynecol. Scand., 2000, 79: 8-14; W. Cheong Leung et al., Prenat. Diagn., 2001, 21: 327-332; J. Pepperberg et al., Prenat. Diagn., 2001, 21: 293-301; S. Weremowicz et al., Prenat. Diagn., 2001, 21: 262-269; and R. Sawa et al., J. Obstet. Gynaecol. Res., 2001, 27: 41-47).

As used herein, the term “Spectral Karyotyping or SKY”, refers to a molecular cytogenetic technique that allows the simultaneous visualization of all chromosomes in different colors, which considerably facilitates karyotype analysis. SKY involves the preparation of a library of short sequences of single-stranded DNA labeled with spectrally distinguishable fluorescent dyes. Each of the individual probes in this DNA library is complementary to a unique region of a chromosome, while together all the probes make up a collection of DNA that is complementary to all of the chromosomes within the genome. After in situ hybridization, the measurement of defined emission spectra by spectral imaging allows for the definitive discernment of all human chromosomes in different colors and the detection of chromosomal abnormalities, such as translocations, chromosomal breakpoints, and rearrangements. For more details about the SKY technique and its use in establishing karyotypes, see, for example, E. Shrock et al., Hum. Genet., 1997, 101: 255-262; I. B. Van den Veyver and B. B. Roa, Curr. Opin. Obstet. Gynecol., 1998, 10: 97-103; M. C. Phelan et al., Prenatal Diagn., 1998, 18: 1174-1180; B. R. Haddad et al., Hum. Genet., 1998, 103: 619-625; and B. Peschka et al., Prenatal. Diagn., 1999, 19: 1143-1149.

The terms “comparative genomic hybridization or CGH” and “metaphase comparative genomic hybridization or metaphase CGH” are used herein interchangeably. They refer to a molecular cytogenetic technique that involves differentially labeling a test DNA and normal reference DNA with fluorescent dyes, co-hybridizing the two labeled DNA samples to normal metaphase chromosome spreads, and visualizing the two hybridized DNAs by fluorescence. The ratio of the intensity of the two fluorescent dyes along a certain chromosome or chromosomal region reflects the relative copy number (i.e., abundance) of the respective nucleic acid sequences in the two samples. A CGH analysis provides a global overview of gains and losses of genetic material throughout the whole genome.

As used herein, the term “statistically significant number” refers to a number of samples (analyzed or to be analyzed) that is large enough to provide reliable data.

The terms “labeled”, “labeled with a detectable agent” and “labeled with a detectable moiety” are used herein interchangeably. They are used to specify that a nucleic acid molecule or individual nucleic acid segments from a sample can be visualized, for example, following binding (i.e., hybridization) to genetic probes. In hybridization methods, samples of nucleic acid segments may be detectably labeled before the hybridization reaction or a detectable label may be selected that binds to the hybridization product. Preferably, the detectable agent or moiety is selected such that it generates a signal which can be measured and whose intensity is related to the amount of hybridized nucleic acids. In array-based methods, the detectable agent or moiety is also preferably selected such that it generates a localized signal, thereby allowing spatial resolution of the signal from each spot on the array. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, colorimetric labels, magnetic labels, and haptens. Detectable moieties can also be biological molecules such as molecular beacons and aptamer beacons.

The terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, R. P. Haugland, “Molecular Probes: Handbook of Fluorescent Probes and Research Chemicals 1992-1994”, 5^{th} Ed., 1994, Molecular Probes, Inc.). In choosing a fluorophore, it is preferred that the fluorescent molecule absorbs light and emits fluorescence with high efficiency (i.e., high molar absorption coefficient and fluorescence quantum yield, respectively) and is photostable (i.e., it does not undergo significant degradation upon light excitation within the time necessary to perform the analysis).

As used herein, the term “computer-assisted imaging system” refers to a system capable of acquiring fluorescence images that can be used to analyze an array after hybridization and to obtain a fluorescence image of the array after hybridization. A computer-assisted imaging system is composed of a hardware, which may comprise an illumination source (such as a laser), a CCD (i.e., charge coupled device) camera, a set of filters, and a computer.

As used herein, the term “computer-assisted image analysis system” refers to a system that can be used to analyze a fluorescence image of an array after hybridization, to interpret data imaged from the array and to display results as fluorescence intensity as a function of genomic locus. A computer-assisted image analysis system may comprise a computer with a software for fluorescence quantitation and fluorescence ratio determination at discrete spots on arrays.

The term “computer” is herein used in its broadest general context. The methods of the invention can be practiced using any computer and in conjunction with any known software or methodology. The computer can further include any form of memory elements, such as dynamic random access memory, flash memory or the like, or mass storage such as magnetic disc optional storage.

### Detailed Description of Certain Preferred Embodiments

The present invention provides systems for assessing gene expression in a living human fetus. Among other things, the inventive systems allow (1) establishment of baseline levels of mRNA expression in karyotypically and developmentally normal fetuses at different gestational ages, (2) establishment of developmental gene expression patterns for karyotypically and developmentally normal fetuses, and (3) identification of novel genes that are abnormally expressed in fetuses with chromosomal aberrations or developmental anomalies, or in fetuses affected with other types of clinical conditions.

The present invention encompasses the discovery that amniotic fluid is a rich source of fetal RNA and relates to methods of isolation and analysis of amniotic fluid fetal RNA. In particular, systems are described that allow for the determination of a fetus's health, growth and development, and for the prenatal diagnosis of a variety of diseases and conditions.

I. Fetal RNA from Amniotic Fluid

In one aspect, the present invention provides isolated amniotic fluid fetal RNA. As mentioned above, the present invention encompasses the recognition, by the inventors, that, notwithstanding the well-known instability of RNA, fetal RNA survives in amniotic fluid in amounts and condition appropriate for analysis.

Amniotic Fluid Sample

Practicing the methods of the invention involves providing a sample of amniotic fluid obtained from a pregnant woman. Amniotic fluid is generally collected by amniocentesis, in which a long needle is inserted in the mother's lower abdomen into the amniotic cavity inside the uterus to withdraw a certain volume of amniotic fluid.

For prenatal diagnosis, most amniocenteses are performed between the 14^{th} and 20^{th} weeks of pregnancy and the volume of amniotic fluid withdrawn is about 10 to 30 mL. Traditionally, the most common indications for amniocentesis include: advanced maternal age (typically set, in the US, at 35 years or more at the estimated time of delivery), previous child with a birth defect or genetic disorder, parental chromosomal rearrangement, family history of late-onset disorders with genetic components, recurrent miscarriages, positive maternal serum screening test (Multiple Marker Screening) documenting increased risk of fetal neural tube defects and/or fetal chromosomal abnormality, and abnormal fetal ultrasound examination (for example, revealing signs known to be associated with fetal aneuploidy). However, the amount and type of information that may be obtained from an amniotic fluid sample according to the present invention may support a change in standard operating procedure, such that amniocentesis is considered or performed in any pregnancy.

Amniocentesis is also performed for therapeutic purposes. In such cases, large amounts of amniotic fluid (>1 L) are removed (amnioreduction) to correct polyhydramnios (i.e., an excess of amniotic fluid surrounding the fetus). Polyhydramnios can represent a danger because of an increased risk of premature rupture of the membranes, and may also be a sign of birth defect or other medical problems such as gestational diabetes or fetal hydrops. Polyhydramnios is also observed in multiple gestations. Twin-to-twin transfusion (TTT) syndrome, is defined sonographically as the combined presence of an excess of amniotic fluid in one sac and an insufficiency of amniotic fluid in the other sac. In TTT syndrome, the goal of the amnioreduction is to attempt to decrease the likelihood of miscarriage or preterm labor by reducing the amniotic fluid volume in the sac of the recipient twin.

In the context of the present invention, samples of amniotic fluid may be obtained after standard or therapeutic amniocentesis. In conventional amniocentesis procedures, fetal cells present in the amniotic fluid are isolated by centrifugation and grown in culture for chromosome analysis, biochemical analysis, and/or molecular biological analysis. Centrifugation also produces a supernatant sample (herein termed “remaining amniotic material”), which is usually stored at −20° C. as a back-up in case of assay failure. Aliquots of this supernatant may also be used for additional assays such as determination of alpha-fetoprotein and acetyl cholinesterase levels. After a certain period of time, the frozen supernatant sample is typically discarded. In amnioreductions, the entire sample of amniotic fluid withdrawn is discarded. The standard protocol followed by the Cytogenetics Laboratory at Tufts-New England Medical Center (Boston, Mass.), which provides the Applicants with fresh and frozen samples of amniotic fluid (from therapeutic amniocenteses) and fresh samples of remaining amniotic material (from diagnostic amniocenteses) is described in detail in the Examples section.

Isolation of Fetal RNA

Fetal RNA for use in the methods of the present invention is isolated from a sample of amniotic fluid obtained from a pregnant woman. The isolation may be carried out by any suitable method of RNA isolation or extraction.

In preferred embodiments, fetal RNA is obtained by treating a sample of amniotic fluid, such that fetal RNA present in the sample of amniotic fluid is extracted. In certain embodiments, fetal RNA is extracted after removal of substantially all or some of the cell populations present in the sample of amniotic fluid. The cell populations may be removed from the amniotic fluid by any suitable method, for example, by centrifugation. More than one centrifugation steps may be performed to ensure that substantially all cell populations have been removed. Preferably, the cell populations are removed within two hours of obtaining the sample of amniotic fluid. More preferably, the cell populations are removed immediately after obtaining the sample of amniotic fluid.

When substantially all cell populations are removed from the sample of amniotic fluid, amniotic fluid fetal RNA consists essentially of cell-free fetal RNA. When extracted from a sample of remaining amniotic material obtained by centrifugation, fetal RNA comprises cell-free fetal RNA as well as fetal RNA from the cells still present in the remaining material.

Fetal RNA may also be obtained by isolating cells from the sample of amniotic fluid, optionally cultivating these isolated cells, and extracting RNA from the cells. In such cases, amniotic fluid fetal RNA consists essentially of fetal RNA from the cultured cells.

As mentioned above, before isolation or extraction of fetal RNA, the sample of remaining amniotic material may be frozen and stored for a certain period of time under suitable storage conditions (e.g., at −80° C.). Before freezing, an RNase inhibitor, which prevents degradation of fetal RNA by RNases (i.e., ribonucleases), may be added to the sample. Preferably, the RNase inhibitor is added within two hours of obtaining the sample of remaining amniotic material. More preferably, the RNase inhibitor is added immediately after obtaining the sample of remaining amniotic material. Before RNA extraction, the frozen sample is thawed at 37° C. and mixed with a vortex. Another centrifugation may be performed at that time to remove any cell populations still present in the amniotic fluid and to ensure that the RNA extracted is truly extracellular.

The most commonly used RNase inhibitor is a natural protein derived from human placenta that specifically (and reversibly) binds RNases (P. Blackburn et al., J. Biol. Chem., 1977, 252: 5904-5910). RNase inhibitors are commercially available, for example, from Ambion (Austin, Tex.; as SUPERase-In™), Promega, Inc. (Madison, Wis.; as rRNasin® Ribonuclease Inhibitor) and Applied Biosystems (Framingham, Mass.). In general, precautions for preventing RNases contaminations in RNA samples, which are well known in the art and include the use of gloves, of certified RNase-free reagents and ware, of specifically treated water and of low temperatures, as well as routine decontamination and the like, are used in the practice of the methods of the invention.

Isolating fetal RNA may include treating the remaining amniotic material such that fetal RNA present in the remaining amniotic material is extracted and made available for analysis. Any suitable isolation method that results in extracted amniotic fluid fetal RNA may be used in the practice of the invention. In order to get the most accurate assessment of the fetus, it is desirable to minimize artifacts from manipulation processes. Therefore, the number of extraction and modification steps is preferably kept as low as possible.

Methods of RNA extraction are well known in the art (see, for example, J. Sambrook et al., “Molecular Cloning: A Laboratory Manual” 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors (J. M. Chirgwin et al., Biochem., 1979, 18: 5294-5299). Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations (see, for example, V. Glisin et al., Biochem., 1974, 13: 2633-2637; D. B. Stern and J. Newton, Meth. Enzymol., 1986, 118: 488).

In certain methods of the invention, for example those in which amniotic fluid fetal RNA is submitted to a gene-expression analysis, it may be desirable to isolate mRNA from total RNA in order to allow the detection of even low level messages (B. Alberts et al., “Molecular Biology of the Cell”, 1994 (3^{rd} Ed.), Garland Publishing, Inc.: New York, N.Y.).

Purification of mRNA from total RNA typically relies on the poly(A) tail present on most mature eukaryotic mRNA species. Several variations of isolation methods have been developed based on the same principle. In a first approach, a solution of total RNA is passed through a column containing oligo(dT) or d(U) attached to a solid cellulose matrix in the presence of high concentrations of salts to allow the annealing of the poly(A) tail to the oligo(dT) or d(U). The column is then washed with a lower salt buffer to remove and release the poly(A) mRNAs. In a second approach, a biotinylated oligo(dT) primer is added to the solution of total RNA and used to hybridize to the 3′ poly(A) region of the mRNAs. The hybridization products are captured and washed at high stringency using streptavidin coupled to paramagnetic particles and a magnetic separation stand. The mRNA is eluted from the solid phase by the simple addition of ribonuclease-free deionized water. Other approaches do not require the prior isolation of total RNA. For example, uniform, superparamagnetic, polystyrene beads with oligo(dT) sequences covalently bound to the surface may be used to isolate mRNA directly by specific base pairing between the poly(A) residues of mRNA and the oligo(dT) sequences on the beads. Furthermore, the oligo(dT) sequence on the beads may also be used as a primer for the reverse transcriptase to subsequently synthesize the first strand of cDNA. Alternatively, new methods or improvements of existing methods for total RNA or mRNA isolation, preparation and/or purification may be devised by one skilled in the art and used in the practice of the methods of the invention.

Numerous different and versatile kits can be used to extract RNA (i.e., total RNA or mRNA) from bodily fluids and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), Dynal Biotech Inc. (Lake Success, N.Y.), Epicentre Technologies (Madison, Wis.), Gentra Systems, Inc. (Minneapolis, Minn.), GIBCO BRL (Gaithersburg, Md.), Invitrogen Life Technologies (Carlsbad, Calif.), MicroProbe Corp. (Bothell, Wash.), Organon Teknika (Durham, N.C.), Promega, Inc. (Madison, Wis.) and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

As described in the Examples section, the Applicants have extracted and purified RNA from remaining amniotic material in the presence of synthetic poly A-RNA as carrier using the Qiagen Viral RNA mini kit and the vacuum protocol. The lowest volumes of remaining amniotic fluid used in these experiments were 420 μL; the highest volumes were 30 mL. The two samples of low volume used in the extraction experiments led to 500 and 1000 pg/mL of purified mRNA, while the two samples of high volume led to 240 and 420 pg/mL of purified mRNA (i.e., 7.2 ng and 12.7 ng, respectively).

Amplification of Extracted Amniotic Fluid Fetal RNA

In certain embodiments, the amniotic fluid fetal RNA is amplified before being analyzed. In other embodiments, before analysis, the amniotic fluid fetal RNA is converted, by reverse-transcriptase, into complementary DNA (cDNA), which, optionally, may, in turn, be converted into complementary RNA (cRNA) by transcription.

Amplification methods are well known in the art (see, for example, A. R. Kimmel and S. L. Berger, Methods Enzymol., 1987, 152: 307-316; J. Sambrook et al., “Molecular Cloning: A Laboratory Manual”, 1989, 2^{nd} Ed., Cold Spring Harbour Laboratory Press: New York; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Standard nucleic acid amplification methods include: polymerase chain reaction (or PCR, see, for example, “PCR Protocols: A Guide to Methods and Applications”, M. A. Innis (Ed.), Academic Press: New York, 1990; and “PCR Strategies”, M. A. Innis (Ed.), Academic Press: New York, 1995); and ligase chain reaction (or LCR, see, for example, U. Landegren et al., Science, 1988, 241: 1077-1080; and D. L. Barringer et al., Gene, 1990, 89: 117-122).

Methods for transcribing RNA into cDNA are also well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase). Other methods include transcription-based amplification system (TAS) (see, for example, D. Y. Kwoh et al., Proc. Natl. Acad. Sci., 1989, 86: 1173-1177), isothermal transcription-based systems such as Self-Sustained Sequence Replication (3SR) (see, for example, J. C. Guatelli et al., Proc. Natl. Acad. Sci., 1990, 87: 1874-1878), and Q-beta replicase amplification (see, for example, J. H. Smith et al., J. Clin. Microbiol., 1997, 35: 1477-1491; and J. L. Burg et al., Mol. Cell. Probes, 1996, 10: 257-271).

The cDNA products resulting from these reverse transcriptase methods may serve as templates for multiple rounds of transcription by the appropriate RNA polymerase (for example, by nucleic acid sequence based amplification or NASBA, see, for example, T. Kievits et al, J. Virol. Methods, 1991, 35: 273-286; and A. E. Greijer et al., J. Virol. Methods, 2001, 96: 133-147). Transcription of the cDNA template rapidly amplifies the signal from the original target mRNA.

These methods as well as others (either known or newly devised by one skilled in the art) may be used in the practice of the invention.

A detailed description of the conversion of amniotic fluid fetal RNA into cRNA by conversion of total extracted RNA into cDNA using a T7-oligo dT primer, synthesis of the second strand of cDNA, purification of the resulting double-stranded cDNA, conversion of the double-stranded cDNA into cRNA by in vitro transcription following the Ambion MEGAscript protocol and purification of the transcripts using RNAeasy columns from Qiagen, can be found in the Examples section

Amplification can also be used to quantify the amount of extracted fetal RNA (see, for example, U.S. Pat. No. 6,294,338). Alternatively or additionally, amplification using appropriate oligonucleotide primers can be used to label cell-free fetal RNA prior to analysis (see below). Suitable oligonucleotide amplification primers can easily be selected and designed by one skilled in the art.

Labeling of Amniotic Fluid Fetal RNA

In certain preferred embodiments, amniotic fluid fetal RNA (for example, after amplification, or after conversion to cDNA or cRNA) is labeled with a detectable agent or moiety before being analyzed. The role of a detectable agent is to facilitate detection of fetal RNA or to allow visualization of hybridized nucleic acid fragments (e.g., nucleic acid fragments bound to genetic probes). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected to reduce steric hindrance, or to confer other useful or desired properties to the resulting labeled molecules (see, for example, E. S. Mansfield et al., Mol. Cell. Probes, 1995, 9: 145-156).

Methods for labeling nucleic acid molecules are well-known in the art. For a review of labeling protocols, label detection techniques and recent developments in the field, see, for example, L. J. Kricka, Ann. Clin. Biochem., 2002, 39: 114-129; R. P. van Gijlswijk et al., Expert Rev. Mol. Diagn., 2001, 1: 81-91; and S. Joos et al., J. Biotechnol., 1994, 35: 135-153. Standard nucleic acid labeling methods include: incorporation of radioactive agents, direct attachment of fluorescent dyes (see, for example, L. M. Smith et al., Nucl. Acids Res., 1985, 13: 2399-2412) or of enzymes (see, for example, B. A. Connoly and P. Rider, Nucl. Acids. Res., 1985, 13: 4485-4502); chemical modifications of nucleic acid fragments making them detectable immunochemically or by other affinity reactions (see, for example, T. R. Broker et al., Nucl. Acids Res., 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res., 1983, 11: 6167-6184; D. J. Brigati et al., Virol., 1983, 126: 32-50; P. Tchen et al., Proc. Natl. Acad. Sci. USA, 1984, 81: 3466-3470; J. E. Landegent et al., Exp. Cell Res., 1984, 15: 61-72; and A. H. Hopman et al., Exp. Cell Res., 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol., 1996, 5: 223-232). More recently developed nucleic acid labeling systems include, but are not limited to: ULS (Universal Linkage System; see, for example, R. J. Heetebrij et al., Cytogenet. Cell. Genet., 1999, 87: 47-52), photoreactive azido derivatives (see, for example, C. Neves et al., Bioconjugate Chem., 2000, 11: 51-55), and alkylating agents (see, for example, M. G. Sebestyen et al., Nat. Biotechnol., 1998, 16: 568-576).

Any of a wide variety of detectable agents can be used in the practice of the present invention. Suitable detectable agents include, but are not limited to: various ligands, radionuclides (such as, for example, ³²p, ³⁵s, ³H, ¹⁴C, 125I, ¹³¹I and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain embodiments, amniotic fluid fetal RNA (after amplification, or conversion to cDNA or cRNA) is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy 5™, Texas red, FITC, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore, see, for example, C. S. Chen et al., J. Org. Chem., 2000, 65: 2900-2906; C. S. Chen et al., J. Biochem. Biophys. Methods, 2000, 42: 137-151; U.S. Pat. Nos. 4,774,339; 5,187,288; 5,227,487; 5,248,782; 5,614,386; 5,994,063; and 6,060,324), and equivalents, analogues, derivatives or combinations of these molecules. Similarly, methods and materials are known for linking or incorporating fluorescent dyes to biomolecules such as nucleic acids (see, for example, R. P. Haugland, “Molecular Probes: Handbook of Fluorescent Probes and Research Chemicals 1992-1994”, 5^{th} Ed., 1994, Molecular Probes, Inc.). Fluorescent labeling dyes as well as labeling kits are commercially available from, for example, Amersham Biosciences, Inc. (Piscataway, N.J.), Molecular Probes, Inc. (Eugene, Oreg.), and New England Biolabs, Inc. (Berverly, Mass.).

Favorable properties of fluorescent labeling agents to be used in the practice of the invention include high molar absorption coefficient, high fluorescence quantum yield, and photostability. Preferred labeling fluorophores exhibit absorption and emission wavelengths in the visible (i.e., between 400 and 750 nm) rather than in the ultraviolet range of the spectrum (i.e., lower than 400 nm).

In other embodiments, amniotic fluid fetal RNA (for example, after amplification or conversion to cDNA or cRNA) is made detectable through one of the many variations of the biotin-avidin system, which are well known in the art. Biotin RNA labeling kits are commercially available, for example, from Roche Applied Science (Indianapolis, Ind.) and Perkin Elmer (Boston, Mass.).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and allowing emission of a fluorescence signal. The use of molecular beacons as detectable moieties is well-known in the art (see, for example, D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (see, for example, O. K. Kaboev et al., Nucleic Acids Res., 2000, 28: E94; R. Yamamoto et al., Genes Cells, 2000, 5: 389-396; N. Hamaguchi et al., Anal. Biochem., 2001, 294: 126-131; S. K. Poddar and C. T. Le, Mol. Cell. Probes, 2001, 15: 161-167).

A “tail” of normal or modified nucleotides may also be added to nucleic acid fragments for detectability purposes. A second hybridization with nucleic acid complementary to the tail and containing a detectable label (such as, for example, a fluorophore, an enzyme or bases that have been radioactively labeled) allows visualization of the nucleic acid fragments bound to the array (see, for example, system commercially available from Enzo Biochem Inc., New York, N.Y.).

The selection of a particular nucleic acid labeling technique will depend on the situation and will be governed by several factors, such as the ease and cost of the labeling method, the quality of sample labeling desired, the effects of the detectable moiety on the hybridization reaction (e.g., on the rate and/or efficiency of the hybridization process), the nature of the detection system to be used, the nature and intensity of the signal generated by the detectable label, and the like.

II. Analysis of Fetal RNA from Amniotic Fluid

The practice of the methods of the invention includes analyzing amniotic fluid fetal RNA to obtain information regarding the fetal RNA. In certain embodiments, analyzing the amniotic fluid fetal RNA comprises determining the quantity, concentration or sequence composition of fetal RNA.

Amniotic fluid fetal RNA may be analyzed by any of a variety of methods. Methods of analysis of RNA are well-known in the art (see, for example, J. Sambrook et al., “Molecular Cloning: A Laboratory Manual”, 1989, 2^{nd} Ed., Cold Spring Harbour Laboratory Press: New York, N.Y.; and “Short Protocols in Molecular Biology”, 2002, F. M. Ausubel (Ed.), 5^{th} Ed., John Wiley & Sons).

For example, the quantity and concentration of fetal RNA extracted from amniotic fluid may be evaluated by UV spectroscopy, wherein the absorbance of a diluted RNA sample is measured at 260 and 280 nm (W. W. Wilfinger et al., Biotechniques, 1997, 22: 474-481). Quantitative measurements may also be carried out using certain fluorescent dyes, such as, for example, RiboGreen® (commercially available from Molecular Probes, Eugene, Oreg,), which exhibit a large fluorescence enhancement when bound to nucleic acids. RNA labeled with these fluorescent dyes can be detected using standard fluorometers, fluorescence microplate reader or filter fluorometers.

Amniotic fluid fetal RNA may also be analyzed through sequencing. For example, RNase T1, which cleaves single-stranded RNA specifically at the 3′-side of guanosine residues in a two-step mechanism, may be used to digest denatured RNA. Partial digestion of 3′ or 5′ labeled RNA with this enzyme thus generates a ladder of G residues. The cleavage can be monitored by radioactive (M. Ikehara et al., Proc. Natl. Acad. Sci. USA, 1986, 83: 4695-4699) or photometric (H. P. Grunert et al., Protein Eng., 1993, 6: 739-744) detection systems, by zymogram assay (J. Bravo et al., Anal. Biochem., 1994, 219: 82-86), agar diffusion test (R. Quaas et al., Nucl. Acids Res., 1989, 17: 3318), lanthan assay (C. B. Anfinsen et al., J. Biol. Chem., 1954, 207: 201-210) or methylene blue test (T. Greiner-Stoeffele et al., Anal. Biochem., 1996, 240: 24-28) or by fluorescence correlation spectroscopy (K. Korn et al., Biol. Chem., 2000, 381: 259-263).

Since the properties and biochemical role of an RNA molecule are determined not only by the RNA sequence but also by its folded structure, analysis of amniotic fluid fetal RNA may also include determination of the RNA structure. Methods for RNA structure analysis are known in the art (see, for example, J. N. Vournakis et al., Gene Amplif. Anal., 1981, 2: 267-298; G. Knapp, Methods Enzymol., 1989, 180: 192-212; A. E. Walter et al., Proc. Natl. Acad. Sci. USA, 1994, 91: 9218-9222). Such analyses may be performed using sequence selective ribonucleases, for example RNase A (which cleaves 3′ to single-stranded C and U residues), RNase V1 (which preferentially cleaves between nucleotides in double-stranded regions of the RNA) and RNase T1.

Other methods for analyzing amniotic fluid fetal RNA include northern blots, wherein the components of the RNA sample being analyzed are resolved by size prior to detection thereby allowing identification of more than one species simultaneously, and slot/dot blots, wherein unresolved mixtures are used.

In certain embodiments, analyzing the amniotic fluid fetal RNA comprises submitting the extracted RNA to a gene-expression analysis. Preferably, this includes the simultaneous analysis of multiple genes.

For example, amniotic fluid fetal RNA analysis may include detecting the presence of a fetal RNA transcribed from the Y chromosome, of a fetal RNA transcribed from a gene or other DNA sequences inherited from either the father or the mother, or of a fetal RNA transcribed from a gene known to be associated with a clinical condition.

For example, the amniotic fluid fetal RNA analysis may include detection of Y-chromosome-specific zinc finger protein (ZFY) mRNA (see, for example, D. C. Page et al., Cell, 1987, 51: 1091-1104; and M. S. Palmer et al., Proc. Natl. Acad. Sci. USA, 1990, 87: 1681-1685). The results of such analyses will lead to identification of fetal gender.

The amniotic fluid fetal RNA analysis may include detection of the presence of RNA transcribed from genes on chromosome 6. Human chromosome 6 is known for encoding the Major Histocompatibility Complex (MHC) which is essential to the immune response. In particular, the analysis may include detection of the presence of RNA transcribed from HLA-G, which is a non-classical human leukocyte antigen expressed primarily in fetal tissues at the maternal-fetal interface (A. Ishitani and D. E. Geraghty, Proc. Natl. Acad. Sci. USA, 1992, 89: 3947-3951; S. E. Hiby et al., Tissue Antigens, 1999, 53: 1-13). Several studies have suggested that HLA-G may be involved in interactions that are critical in establishing and/or maintaining pregnancy (N. Hara et al., Am. J. Reprod. Immunol., 1996, 36: 349-358; K. H. Lim et al., Am. J. Pathol., 1997, 151: 1809-1818; K. A. Pfeiffer et al., Mol. Hum. Reprod., 2001, 7: 373-378). Detection of the presence of RNA transcribed from HLA-G may, for example, lead a health provider to advise precautions against miscarriage.

The amniotic fluid fetal RNA analysis may include detection and determination of expression levels of surfactant genes as a way of monitoring fetal lung development.

Detection of the presence of RNA transcribed from a gene known to be associated with a disease or condition may be used to provide a prenatal diagnosis.

In analyses carried out to detect the presence or absence of RNA transcribed from a specific gene, the detection may be performed by any of a variety of physical, immunological and biochemical methods. Such methods are well-known in the art, and include, for example, protection from enzymatic degradation such as S1 analysis and RNase protection assays, in which hybridization to a labeled nucleic acid probe is followed by enzymatic degradation of single-stranded regions of the probe and analysis of the amount and length of probe protected from degradation. The TaqMan assay, a quenched fluorescent dye system, may also be used to quantitate targeted mRNA levels (see, for example K. J. Livak et al., PCR Methods Appl., 1995, 4: 357-362).

Other methods are based on the analysis of cDNA derived from mRNA, which is less sensitive to degradation than RNA and therefore easier to handle. These methods include, but are not limited to, sequencing cDNA inserts of an expressed sequence tag (EST) clone library (see, for example, M. D. Adams et al., Science, 1991, 252: 1651-1656) and serial analysis of gene expression (or SAGE), which allows quantitative and simultaneous analysis of a large number of transcripts (see, for example, U.S. Pat. No. 5,866,330; V. E. Velculescu et al., Science, 1995, 270: 484-487; and Zhang et al., Science, 1997, 276: 1268-1272). These two methods survey the whole spectrum of mRNA in a sample rather than focusing on a predetermined set.

Other methods of analysis of cDNA derived from mRNA include reverse transcriptase-mediated PCR (RT-PCR) gene expression assays. These methods are directed at specific target gene products and allow the qualitative (non-quantitative) detection of transcripts of very low abundance (see, for example, S. Su et al., BioTechniques, 1997, 22: 1107-1113). A variation of these methods, called competitive RT-PCR, in which a known amount of exogenous template is added as internal control, has been developed to allow quantitative measurements (see, for example, M. Beker-Andre and K. Hahlbrock, Nucl. Acids Res., 1989, 17: 9437-9346; A. M. Wang et al., Proc. Natl. Acad. Sci. USA, 1989, 86: 9717-9721; G. Gilliland et al., Proc. Natl. Acad. Sci. USA, 1990, 87: 2725-2729).

mRNA analysis may also be performed by differential display reverse transcriptase PCR (DDRT-PCR; see, for example, P. Liang and A. B. Pardee, Science, 1992, 257: 967-971) or RNA arbitrarily primed PCR (RAP-CPR; see, for example, J. Welsh et al., Nucl. Acids Res., 1992, 20: 4965-4970). In these methods, RT-PCR fingerprint profiles of transcripts are generated by random priming and differentially expressed genes appear as changes in the fingerprint profiles between two samples. Identification of a differentially expressed gene requires further manipulation (i.e., the appropriate band of the gel must be excised, subcloned, sequenced and matched to a gene in a sequence database).

III. Array-Based Gene Expression Analysis of Amniotic Fetal RNA

In certain embodiments, the methods of the invention include submitting amniotic fluid fetal RNA to an array-based gene expression analysis.

Array-Based Gene Expression Analysis

Traditional molecular biology methods, such as most of those described above, typically assess one gene per experiment, which significantly limits the overall throughput and prevents gaining a broad picture of gene function. Technologies based on DNA array or microarray (also called gene expression microarray), which were developed more recently, offer the advantage of allowing the monitoring of thousands of genes simultaneously through identification of sequence (gene/gene mutation) and determination of gene expression level (abundance) of genes (see, for example, A. Marshall and J. Hodgson, Nature Biotech., 1998, 16: 27-31; G. Ramsay, Nature Biotech., 1998, 16: 40-44; R. Ekins and R. W. Chu, Trends in Biotech., 1999, 17: 217-218; and D. J. Lockhart and E. A. Winzeler, Nature, 2000, 405: 827-836).

The principle of a gene expression experiment is simple: labeled cDNA or cRNA targets derived from the mRNA of an experimental sample are hybridized to nucleic acid probes immobilized to a solid support. By monitoring the amount of label associated with each DNA location, it is possible to infer the abundance of each mRNA species represented.

There are two standard types of DNA microarray technology in terms of the nature of the arrayed DNA sequence. In the first format, probe cDNA sequences (typically 500 to 5,000 bases long) are immobilized to a solid surface and exposed to a plurality of targets either separately or in a mixture. In the second format, oligonucleotides (typically 20-80-mer oligos) or peptide nucleic acid (PNA) probes are synthesized either in situ (i.e., directly on-chip) or by conventional synthesis followed by on-chip attachment, and then exposed to labeled samples of nucleic acids.

The analyzing step in the methods of the invention can be performed using any of a variety of methods, means and variations thereof for carrying out array-based gene expression analysis. Array-based gene expression methods are known in the art and have been described in numerous scientific publications as well as in patents (see, for example, M. Schena et al., Science, 1995, 270: 467-470; M. Schena et al., Proc. Natl. Acad. Sci. USA, 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Array-based gene-expression methods have been developed and used in medicine and clinical research, for example, in cancer (see, for example, J. DeRisi et al., Nat. Genet., 1996, 14: 457-460; S. Mohr et al., J. Clin. Oncol., 2002, 20: 3165-3175); in breast cancer research (see, for example, the review by C. S. Cooper, Breast Cancer Res., 2001, 3: 158-175); in brain tumors (see, for example, S. B. Hunter and C. S. Moreno, Front Biosci., 2002, 7: c74-c82); in oral cancers (see, for example, R. Todd and D. T. Wong, J. Dent. Res., 2002, 81: 89-97); in islet biology and diabetes research (see, for example, E. Bemal-Mizrachi et al., Diabetes Metab. Res. Rev., 2003, 19: 32-42); in studies of pulmonary fibrosis, asthma, acute lung injury and emphysema (see, for example, M. W. Geraci et al., Respir. Res., 2001, 2: 210-215; and D. Sheppard, Chest, 2002, 121: 21S-25S); in otolaryngology-head and neck surgery (see, for example, M. E. Whipple and W. P. Kuo, Otolaryngol. Head Neck Surg., 2002, 127: 196-204); in brain disorders (see, for example, P. D. Shilling and J. R. Kelsoe, Pharmacogenomics, 2002, 3: 31-45); in renal biology and medicine (see, for example, M. Eikmans et al., Kidney Int., 2002, 62: 1125-1135; C. D. Cohen and M. Kretzler, Nephron., 2002, 92: 522-528; and E. P. Bottinger et al., Exp. Nephrol., 2002, 10: 93-101); in hematology (see, for example, J. Walker et al., Curr. Opin. Hematol., 2002, 9: 23-29); in pharmacogenomic research (see, for example, M. Srivastava et al., Mol. Med., 1999, 5: 753-767; and P. E. Blower et al., Pharmacogen. J., 2002, 2: 259-271); in drug discovery (see, for example, C. Debouk and P. N. Goodfellow, Nat. Genet., 1999, 21: 48-50; and A. Butte, Nat. Rev. Drug Discov., 2002, 1: 951-960); as a tool for the discovery of novel genes involved in psychiatric disorders (see, for example, A. B. Niculescu and J. R. Kelsoe, Ann. Med., 2001, 33: 263-271); to study gene expression in host cells infected with viruses (see, for example, J. H. Nam et al., Acta Virol., 2002, 46: 141-146); to study gene expression in the nervous system (see, for example, T. J. Sendera et al., Neurochem. Res., 2002, 27: 1005-1026; R. S. Griffin et al., Genome Biol., 2003, 4: 105), and to elucidate and interpret the mechanistic roles of genes in pathogenesis of infectious diseases (see, for example, M. Kato-Maeda et al., Cell Microbiol., 2001, 3: 713-719).

In the practice of the present invention, these methods as well as other methods known in the art for carrying out array-based gene expression analysis may be used as described or modified such that they allow fetal mRNA levels of gene expression to be evaluated.

Other methods of the invention for establishing gene expression in a fetus, comprise steps of: providing a test sample of amniotic fluid fetal RNA, wherein the fetal RNA comes from a sample of amniotic fluid obtained from a pregnant woman, and wherein the test sample comprises a plurality of nucleic acid segments labeled with a detectable agent; providing a gene-expression array comprising a plurality of genetic probes, wherein each genetic probe is immobilized to a discrete spot on a substrate surface to form the array; contacting the array with the test sample under conditions wherein the nucleic acid segments in the sample specifically hybridize to the genetic probes on the array; determining the binding of individual nucleic acid segments of the test sample to individual genetic probes immobilized on the array to obtain a binding pattern; and based on the binding pattern obtained, establishing a gene expression pattern for the fetus.

Test Sample

Preferably, amniotic fluid fetal RNA to be analyzed by an array-based gene expression method is isolated from a sample of amniotic fluid as described above. A test sample of amniotic fluid fetal RNA to be used in the methods of the invention includes a plurality of nucleic acid fragments labeled with a detectable agent.

The extracted fetal RNA may be amplified, reverse-transcribed, labeled, fragmented, purified, concentrated and/or otherwise modified prior to the gene-expression analysis. Techniques for the manipulation of nucleic acids are well-known in the art, see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons.

In certain preferred embodiments, in order to improve the resolution of the array-based gene expression analysis, the nucleic acid fragments of the test sample are less then about 500 bases long, preferably less than about 200 bases long. The use of small fragments significantly increases the reliability of the detection of small differences or the detection of unique sequences.

Methods of RNA fragmentation are known in the art and include: treatment with ribonucleases (e.g., RNase T1, RNase V1 and RNase A), sonication (see, for example, P. L. Deininger, Anal. Biochem., 1983, 129: 216-223), mechanical shearing, and the like (see, for example, J. Sambrook et al., “Molecular Cloning: A Laboratory Manual”, 1989, 2^{nd} Ed., Cold Spring Harbour Laboratory Press: New York; P. Tijssen “Hybridization with Nucleic Acid Probes—Laboratory Techniques in Biochemistry and Molecular Biology (Parts I and II)”, 1993, Elsevier Science; C. P. Ordahl et al., Nucleic Acids Res. 1976, 3: 2985-2999; P. J. Oefner et al., Nucleic Acids Res., 1996, 24: 3879-3886; Y. R. Thorstenson et al., Genome Res., 1998, 8: 848-855). Random enzymatic digestion of the RNA leads to fragments containing as low as 25 to 30 bases.

Fragment size of the nucleic acid segments in the test sample may be evaluated by any of a variety of techniques, such as, for example, electrophoresis (see, for example, B. A. Siles and G. B. Collier, J. Chromatogr. A, 1997, 771: 319-329) or matrix-assisted laser desorption/ionization time-of-flight mass spectrometry (see, for example, N. H. Chiu et al., Nucl. Acids, Res., 2000, 28: E31).

In the practice of the methods of the invention, the test sample of amniotic fluid fetal RNA is labeled before analysis. Suitable methods of nucleic acid labeling with detectable agents have been described in detail above.

Prior to hybridization, the labeled nucleic acid fragments of the test sample may be purified and concentrated before being resuspended in the hybridization buffer. Microcon 30 columns may be used to purify and concentrate samples in a single step. Alternatively, nucleic acids may be purified using a membrane column (such as a Qiagen column) or sephadex G50 and precipitated in the presence of ethanol.

Methods of preparation of nucleic acid samples for gene-expression array hybridization experiments can easily be performed and/or modified by one skilled in the art.

Gene-Expression Hybridization Arrays

Any of a variety of arrays may be used in the practice of the present invention. Investigators can either rely on commercially available arrays or generate their own. Methods of making and using arrays are well known in the art (see, for example, S. Kern and G. M. Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol., 1998, 8: R171-R174; D. D. Bowtell, Nature Gen., 1999, Supp. 21:25-32; S. J. Watson and H. Akil, Biol Psychiatry., 1999, 45: 533-543; W. M. Freeman et al., Biotechniques, 2000, 29: 1042-1046 and 1048-1055; D. J. Lockhart and E. A. Winzeler, Nature, 2000, 405: 827-836; M. Cuzin, Transfus. Clin. Biol., 2001, 8:291-296; P. P. Zarrinkar et al., Genome Res., 2001, 11: 1256-1261; M. Gabig and G. Wegrzyn, Acta Biochim. Pol., 2001, 48: 615-622; and V. G. Cheung et al., Nature, 2001, 40: 953-958; see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Arrays comprise a plurality of genetic probes immobilized to discrete spots (i.e., defined locations or assigned positions) on a substrate surface. Substrate surfaces for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may preferably be used (see, for example, U.S. Pat. No. 6,063,338).

The presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Array-immobilized genetic probes may be nucleic acids that contain sequences from genes (e.g., from a genomic library), including, for example, sequences that collectively cover a substantially complete genome or a subset of a genome (for example, the array may contain only human genes that are expressed throughout development). Genetic probes may be long cDNA sequences (500 to 5,000 bases long) or shorter sequences (for example, 20-80-mer oligonucleotides). The sequences of the genetic probes are those for which gene expression levels information is desired. Additionally or alternatively, the array may comprise nucleic acid sequences of unknown significance or location. Genetic probes may be used as positive or negative controls (for example, the nucleic acid sequences may be derived from karyotypically normal genomes or from genomes containing one or more chromosomal abnormalities; alternatively or additionally, the array may contain perfect match sequences as well as single base pair mismatch sequences to adjust for non-specific hybridization).

Techniques for the preparation and manipulation of genetic probes are well-known in the art (see, for example, J. Sambrook et al., “Molecular Cloning: A Laboratory Manual”, 1989, 2^{nd} Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5^{th} Ed., John Wiley & Sons).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from about 5 to about 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; about 70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

Alternatively, genetic probes, especially those containing short sequences such as oligonucleotides, are synthesized in vitro by chemical techniques well-known in the art and then immobilized on arrays. Such methods have been described in scientific articles as well as in patents (see, for example, S. A. Narang et al., Meth. Enzymol., 1979, 68: 90-98; E. L. Brown et al., Meth. Enzymol., 1979, 68: 109-151; E. S. Belousov et al., Nucleic Acids Res., 1997, 25: 3440-3444; D. Guschin et al., Anal. Biochem., 1997, 250: 203-211; M. J. Blommers et al., Biochemistry, 1994, 33: 7886-7896; and K. Frenkel et al., Free Radic. Biol. Med., 1995, 19: 373-380; see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′-phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, U. Maskos and E. M. Southern, Nucleic Acids Res., 1992, 20: 1679-1684; R. S. Matson et al., Anal. Biochem., 1995, 224; 110-116; R. J. Lipshutz et al., Nat. Genet., 1999, 21: 20-24; Y. H. Rogers et al., Anal. Biochem., 1999, 266: 23-30; M. A. Podyminogin et al., Nucleic Acids Res., 2001, 29: 5090-5098; Y. Belosludtsev et al., Anal. Biochem., 2001, 292: 250-256).

Oligonucleotide-based arrays have also been prepared by synthesis in situ using a combination of photolitography and oligonucleotide chemistry (see, for example, A. C. Pease et al., Proc. Natl. Acad. Sci. USA, 1994, 91: 5022-5026; D. J. Lockhart et al., Nature Biotech., 1996, 14: 1675-1680; S. Singh-Gasson et al., Nat. Biotechn., 1999, 17: 974-978; M. C. Pirrung et al., Org. Lett., 2001, 3: 1105-1108; G. H. McGall et al., Methods Mol. Biol., 2001, 170; 71-101; A. D. Barone et al., Nucleosides Nucleotides Nucleic Acids, 2001, 20: 525-531; J. H. Butler et al., J. Am. Chem. Soc., 2001, 123: 8887-8894; E. F. Nuwaysir et al., Genome Res., 2002, 12: 1749-1755). The chemistry for light-directed oligonucleotide synthesis using photolabile protected 2′-deoxynucleoside phosphoramites has been developed by Affymetrix Inc. (Santa Clara, Calif.) and is well known in the art (see, for example, U.S. Pat. No. 5,424,186 and 6,582,908).

An alternative to custom arraying of genetic probes is to rely on commercially available arrays and micro-arrays. Such arrays have been developed, for example, by Affymetrix Inc. (Santa Clara, Calif.), Illumina, Inc. (San Diego, Calif.), Spectral Genomics, Inc. (Houston, Tex.), and Vysis Corporation (Downers Grove, Ill.).

As described in the Examples section, the Applicants have used two different gene expression arrays. The first one is the GeneChip Test3 array (developed and commercialized by Affymetrix), which contains a subset of 24 human genes that are expressed throughout development. This array is used to assess the quality and quantity of cRNA for subsequent application to microarray with larger set of human genes. The second array is the Affymetrix gene expression microarray HG-U133A, which contains 22,283 probe elements that represent 14,239 unique genes, wherein in some cases more than one probe element represents a single mRNA transcript. The sequences from which these probe sets were derived were selected from GenBank®, dbEST, and RefSeq. Each probe element consists of at least 22 25-mer oligonucleotide sequences; half of these are perfect match sequences and the other half are single base pair mismatch sequences to adjust for non-specific hybridization. In this array, the hybridization ratio of the cRNA to the perfect/mismatch sequences yields qualitative data on the presence or absence of each unique gene.

Hybridization

In the methods of the invention, the gene expression array is contacted with the test sample under conditions wherein the nucleic acid fragments in the sample specifically hybridize to the genetic probes immobilized on the array.

The hybridization reaction and washing step(s), if any, may be carried out under any of a variety of experimental conditions. Numerous hybridization and wash protocols have been described and are well-known in the art (see, for example, J. Sambrook et al., “Molecular Cloning: A Laboratory Manual”, 1989, 2^{nd} Ed., Cold Spring Harbour Laboratory Press: New York; P. Tijssen “Hybridization with Nucleic Acid Probes—Laboratory Techniques in Biochemistry and Molecular Biology (Part II)”, Elsevier Science, 1993; and “Nucleic Acid Hybridization”, M. L. M. Anderson (Ed.), 1999, Springer Verlag: New York, N.Y.). The methods of the invention may be carried out by following known hybridization protocols, by using modified or optimized versions of known hybridization protocols or newly developed hybridization protocols as long as these protocols allow specific hybridization to take place.

The term “specific hybridization” refers to a process in which a nucleic acid molecule preferentially binds, duplexes, or hybridizes to a particular nucleic acid sequence under stringent conditions. In the context of the present invention, this term more specifically refers to a process in which a nucleic acid fragment from a test sample preferentially binds (i.e., hybridizes) to a particular genetic probe immobilized on the array and to a lesser extent, or not at all, to other immobilized genetic probes of the array. Stringent hybridization conditions are sequence dependent. The specificity of hybridization increases with the stringency of the hybridization conditions; reducing the stringency of the hybridization conditions results in a higher degree of mismatch being tolerated.

The hybridization and/or wash conditions may be adjusted by varying different factors such as the hybridization reaction time, the time of the washing step(s), the temperature of the hybridization reaction and/or of the washing process, the components of the hybridization and/or wash buffers, the concentrations of these components as well as the pH and ionic strength of the hybridization and/or wash buffers.

In certain embodiments, the hybridization and/or wash steps are carried out under very stringent conditions. In other embodiments, the hybridization and/or wash steps are carried out under moderate to stringent conditions. In still other embodiments, more than one washing steps are performed. For example, in order to reduce background signal, a medium to low stringency wash is followed by a wash carried out under very stringent conditions.

As is well known in the art, the hybridization process may be enhanced by modifying other reaction conditions. For example, the efficiency of hybridization (i.e., time to equilibrium) may be enhanced by using reaction conditions that include temperature fluctuations (i.e., differences in temperature that are higher than a couple of degrees). An oven or other devices capable of generating variations in temperatures may be used in the practice of the methods of the invention to obtain temperature fluctuation conditions during the hybridization process.

It is also known in the art that hybridization efficiency is significantly improved if the reaction takes place in an environment where the humidity is not saturated. Controlling the humidity during the hybridization process provides another means to increase the hybridization sensitivity. Array-based instruments usually include housings allowing control of the humidity during all the different stages of the experiment (i.e., pre-hybridization, hybridization, wash and detection steps).

Additionally or alternatively, a hybridization environment that includes osmotic fluctuation may be used to increase hybridization efficiency. Such an environment where the hyper-/hypo-tonicity of the hybridization reaction mixture varies may be obtained by creating a solute gradient in the hybridization chamber, for example, by placing a hybridization buffer containing a low salt concentration on one side of the chamber and a hybridization buffer containing a higher salt concentration on the other side of the chamber.

Highly Repetitive Sequences

In the practice of the methods of the invention, the array is contacted with the test sample under conditions wherein the nucleic acid segments in the sample specifically hybridize to the genetic probes on the array. As mentioned above, the selection of appropriate hybridization conditions will allow specific hybridization to take place. In certain cases, the specificity of hybridization may further be enhanced by inhibiting repetitive sequences.

In certain preferred embodiments, repetitive sequences present in the nucleic acid fragments are removed or their hybridization capacity is disabled. By excluding repetitive sequences from the hybridization reaction or by suppressing their hybridization capacity, one prevents the signal from hybridized nucleic acids to be dominated by the signal originating from these repetitive-type sequences (which are statistically more likely to undergo hybridization). Failure to remove repetitive sequences from the hybridization or to suppress their hybridization capacity results in non-specific hybridization, making it difficult to distinguish the signal from the background noise.

Removing repetitive sequences from a mixture or disabling their hybridization capacity can be accomplished using any of a variety of methods well-known to those skilled in the art. These methods include, but are not limited to, removing repetitive sequences by hybridization to specific nucleic acid sequences immobilized to a solid support (see, for example, O. Brison et al., Mol. Cell. Biol., 1982, 2: 578-587); suppressing the production of repetitive sequences by PCR amplification using adequate PCR primers; or inhibiting the hybridization capacity of highly repeated sequences by self-reassociation (see, for example, R. J. Britten et al., Methods of Enzymol., 1974, 29: 363-418).

Preferably, the hybridization capacity of highly repeated sequences is competitively inhibited by including, in the hybridization mixture, unlabeled blocking nucleic acids. The unlabeled blocking nucleic acids, which are mixed to the test sample before the contacting step, act as a competitor and prevent the labeled repetitive sequences from binding to the highly repetitive sequences of the genetic probes, thus decreasing hybridization background. In certain embodiments, for example when cDNA derived from fetal mRNA is analyzed, the unlabeled blocking nucleic acids are Human Cot-1 DNA. Human Cot-1 DNA is commercially available, for example, from Gibco/BRL Life Technologies (Gaithersburg, Md.).

Binding Detection and Data Analysis

The methods of the invention include determining the binding of individual nucleic acid fragments of the test sample to individual genetic probes immobilized on the array in order to obtain a binding pattern. In array-based gene expression, determination of the binding pattern is carried out by analyzing the labeled array which results from hybridization of labeled nucleic acid segments to immobilized genetic probes.

In certain embodiments, determination of the binding includes: measuring the intensity of the signals produced by the detectable agent at each discrete spot on the array.

Analysis of the labeled array may be carried out using any of a variety of means and methods, whose selection will depend on the nature of the detectable agent and the detection system of the array-based instrument used.

In certain embodiments, the detectable agent comprises a fluorescent dye and the binding is detected by fluorescence. In other embodiments, the sample of amniotic fluid fetal RNA is biotin-labeled and after hybridization to immobilized genetic probes, the hybridization products are stained with a streptavidin-phycoerythrin conjugate and visualized by fluorescence. Analysis of a fluorescently labeled array usually comprises: detection of fluorescence over the whole array, image acquisition, quantitation of fluorescence intensity from the imaged array, and data analysis.

Methods for the detection of fluorescent labels and the creation of fluorescence images are well known in the art and include the use of “array reading” or “scanning” systems, such as charge-coupled devices (i.e., CCDs). Any known device or method, or variation thereof can be used or adapted to practice the methods of the invention (see, for example, Y. Hiraoka et al., Science, 1987, 238: 36-41; R. S. Aikens et al., Meth. Cell Biol., 1989, 29: 291-313; A. Divane et al., Prenat. Diagn., 1994, 14: 1061-1069; S. M. Jalal et al., Mayo Clin. Proc., 1998, 73: 132-137; V. G. Cheung et al., Nature Genet., 1999, 21: 15-19; see also, for example, U.S. Pat. Nos. 5,539,517; 5,790,727; 5,846,708; 5,880,473; 5,922,617; 5,943,129; 6,049,380; 6,054,279; 6,055,325; 6,066,459; 6,140,044; 6,143,495; 6,191,425; 6,252,664; 6,261,776 and 6,294,331).

Commercially available microarrays scanners are typically laser-based scanning systems that can acquire one (or more) fluorescent image (such as, for example, the instruments commercially available from PerkinElmer Life and Analytical Sciences, Inc. (Boston, Mass.), Virtek Vision, Inc. (Ontario, Canada) and Axon Instruments, Inc. (Union City, Calif.)). Arrays can be scanned using different laser intensities in order to ensure the detection of weak fluorescence signals and the linearity of the signal response at each spot on the array. Fluorochrome-specific optical filters may be used during the acquisition of the fluorescent images. Filter sets are commercially available, for example, from Chroma Technology Corp. (Rockingham, Vt.).

Preferably, a computer-assisted imaging system capable of generating and acquiring fluorescence images from arrays such as those described above, is used in the practice of the methods of the invention. One or more fluorescent images of the labeled array after hybridization may be acquired and stored.

Preferably, a computer-assisted image analysis system is used to analyze the acquired fluorescent images. Such systems allow for an accurate quantitation of the intensity differences and for an easier interpretation of the results. A software for fluorescence quantitation and fluorescence ratio determination at discrete spots on an array is usually included with the scanner hardware. Softwares and/or hardwares are commercially available and may be obtained from, for example, BioDiscovery (El Segundo, Calif.), Imaging Research (Ontario, Canada), Affymetrix, Inc. (Santa Clara, Calif.), Applied Spectral Imaging Inc. (Carlsbad, Calif.); Chroma Technology Corp. (Brattleboro, Vt.); Leica Microsystems, (Bannockburn, Ill.); and Vysis Inc. (Downers Grove, Ill.). Other softwares are publicly available (e.g., MicroArray Image Analysis, and Combined Expression Data and Sequence Analysis (http://rana.lbl.gov); D. Y. Chiang et al., Bioinformatics, 2001, 17: 49-55; the system written in R and available through the Bioconductor project (http://www.bioconductor.org); the Java-based TM4 software system available from the Institute for Genomic Research (http://www.tigr.org/software); and the Web-based system developed at Lund University (http://base.thep.lu.se)).

As described in the Examples section, the Applicants have used the software necessary to analyze the Affymetrix microarrays.

Accurate determination of fluorescence intensities requires normalization and determination of the fluorescence ratio baseline (A. Brazma and J. Vilo, FEBS Lett., 2000, 480: 17-24). Data reproducibility may be assessed by using arrays on which genetic probes are spotted in duplicate or triplicate. Baseline thresholds may also be determined using global normalization approaches (M. K. Kerr et al., J. Comput. Biol., 2000, 7: 819-837). Other arrays include a set of maintenance genes which shows consistent levels of expression over a wide variety of tissues and allows the normalization and scaling of array experiments.

In the practice of the methods of the invention, any of a large variety of bioinformatics and statistical methods may be used to analyze data obtained by array-based gene expression analysis. Such methods are well known in the art (for a review of essential elements of data acquisition, data processing, data analysis, data mining and of the quality, relevance and validation of information extracted by different bioinformatics and statistical methods, see, for example, A. Watson et al., Curr. Opin. Biotechnol., 1998, 9: 609-614; D. J. Duggan et al., Nat. Genet., 1999, 21: 10-14; D. E. Bassett et al., Nat. Genet., 1999, 21: 51-55; K. R. Hess et al., Trends Biotechnol., 2001, 19: 463-468; E. Marcotte and S. Date, Brief Bioinform., 2001, 2: 363-374; J. N. Weinstein et al., Cytometry, 2002, 47: 46-49; T. G. Dewey, Drug Discov. Today, 2002, 7: S170-S175; A Butte, Nat. Rev. Drug Discov., 2002, 1: 951-960; J. Tamames et al., J. Biotechnol., 2002, 98: 269-283; Z Xiang et al., Curr. Opin. Drug Discov. Devel., 2003, 6: 384-395.

The procedure followed by the Applicants to extract gene expression information from the data obtained including using the Affymetrix software Data Mining Tool and the internet-based program NetAffx™ is described in the Examples section.

IV. Gene Expression Pattern and Prenatal Diagnosis

The methods of the invention described above may be used to establish gene expression pattern in a fetus, and thereby can provide information that is not available using conventional methods of prenatal diagnosis.

Knowledge of the gene expression profile in a fetus at different gestational ages would provide a much better understanding of the basic genetic mechanisms that underlie normal and abnormal developmental processes in utero, and would allow identification and mapping of genes responsible for specific birth defects, developmental anomalies, and other clinical conditions (G. C. Weston et al., Austr. and New Zeal. J. Obst. Gyn., 2003, 43: 264-272). Furthermore, such knowledge could be used to provide more accurate prenatal diagnosis and could allow the development of novel strategies for the prevention and/or treatment of prenatal physiological and pathological conditions.

Due to the difficulties inherent to research on human embryos, almost nothing is known about genes active in human early development that was directly studied on humans. Traditionally, investigations to gain in-depth insight into developmental gene expression patterns at different human embryonic and fetal stages have relied upon the use of model systems. This approach is justified by the conservation of genes, genetic networks, and developmental pathways across the animal kingdom. Numerous studies, which have focused on lower organisms, in particular the fruit fly Drosophila, have identified families of genes which control early developmental events in a diverse range of multicellular organisms. These genes have been found to be crucial to normal mammalian development and several are known to be responsible for human birth defects. Other studies have established gene expression patterns in the developing zebrafish, xenopus, and mouse (T. S. Tanaka et al., Proc. Natl. Acad. Sci. USA, 2000, 97: 9127-9132). The large amounts of gene-expression data on major model embryos used in developmental biology are now too extensive to be stored in any format other than databases (see, for example, J. B. L. Bard, Sem. Cell. Develop. Biol., 1997, 8: 455-458; R. A. Baldock et al., Sem. Cell. Develop. Biol., 1997, 8: 499-507; D. Davidson et al., Sem. Cel. Develop. Biol., 1997, 8: 509-511; J. B. Bard et al., Genome Res., 1998, 8: 859-863; F. J. Verbeek et al., Int. J. Dev. Biol., 1999, 43: 761-771; J. Streicher et al., Nat. Genet., 2000, 25: 147-152; D. Davidson and R. Baldock, Nat. Rev. Genet., 2001, 2: 409-418; J. Sharpe et al., Science, 2002, 296: 541-545).

Information about the expression of embryonic genes has also been obtained using human individual preimplantation embryos at different stages of preimplantation development (see, for example, Y. Verlinsky et al., Mol. Hum. Reprod., 1998, 4: 571-575; D. M. Gou et al., Gene, 2001, 278: 141-147). This strategy has made possible the identification and isolation of human genes specifically expressed at the different stages of human preimplantation development from the unfertilized oocyte to the blastocyst stage (J. Adjaye et al., J. Assist. Reprod. Genet., 1998, 15: 344-348; C. Holding et al., Mol. Hum. Reprod. 2000, 6: 801-809; M. Monk et al., Reprod. Fertil. Dev., 2001, 13: 51-57).

The methods of the present invention have the advantage of being based on the direct analysis of RNA from living human fetuses. In particular, the methods of the invention can be used to establish baseline levels of mRNA gene expression in karyotypically and developmentally normal male, and normal female fetuses at different gestational ages. Since fetal RNA used in the inventive methods is extracted from amniotic fluid, the gestational period for which gene expression patterns may be established corresponds to that during which amniocentesis has been determined to be “safe”.

The methods of the invention may also be used to establish developmental gene expression patterns for karyotypically and developmentally normal male, and normal female fetuses at different gestational ages (wherein one or more feature(s) of the gene expression patterns is/are correlated to a time or event in the development of the fetus).

Knowledge of the gene expression profile in a fetus at different gestational ages should provide a better understanding of the basic genetic mechanisms that underlie normal developmental processes in utero. Once this knowledge is acquired according to the present invention, genes responsible for specific birth defects, developmental anomalies, and other clinical conditions, can be identified and mapped, which will then lead to a better understanding of the basic genetic mechanisms that underlie abnormal developmental processes in utero.

For example, gene expression profiles obtained using the inventive methods during human nephrogenesis could provide insights into normal kidney development as well as aberrations of this normal process that may result in renal dysplasia or pediatric renal malignancy (Wilms's tumor).

Similarly, gene expression patterns of small for gestational age (SGA) and appropriate for gestational age (AGA) fetuses can be compared using the methods of the invention. Small for gestational age babies usually have birth-weights below the 10^{th} percentile for babies of the same gestational age. SGA may also encompass specific metabolic abnormalities including hypoglycemia, hypothermia, and polycythemia. Low birth-weight babies make a disproportionate contribution to perinatal morbidity and mortality. Furthermore, problems arising after birth and later in life, such as poor cognitive development, neurologic impairment, development of cardiovascular disease, high blood pressure, obstructive lung disease, diabetes, high cholesterol concentrations and renal damage have been reported to be associated with low birth-weight (C. H. D. Fall et al., J. Nutr., 2003, 133: 1747S-1756S).

Similarly, the methods of the invention may be used to analyze fetal RNA extracted from amniotic fluid from fetuses that are undergoing lung maturity testing to determine if a profile of normal or delayed pulmonary gene expression can be established.

The information obtained through the methods of the invention may then be used to provide prenatal diagnosis. More specifically, comparison of the gene expression pattern of a fetus with baseline levels of mRNA gene expression established for karyotypically and developmentally normal fetuses at different gestational ages will allow detection and identification of genes abnormally expressed in the fetus. Therefore, when coupled to amniocentesis, the methods of prenatal diagnosis of the invention will lead to a wealth of information about the fetus that is not currently available using standard technologies.

In addition, novel genes shown by the methods of the invention to be abnormally expressed in specific birth defects, developmental anomalies, or other clinical conditions could also potentially serve as future targets for amplification in maternal blood (thereby eliminating the need for the pregnant woman to undergo amniocentesis).

V-Kits

In another aspect, the present invention provides kits comprising materials useful for carrying out the methods of the invention.

Inventive kits contain some or all of the following components: materials to extract cell-free fetal RNA from a sample of amniotic fluid obtained from a pregnant woman; a gene expression array comprising a plurality of genetic probes, wherein each genetic probe is immobilized to a discrete spot on a substrate surface to form the array; a database comprising baseline levels of mRNA expression established for karyotypically and developmentally normal male, and normal female fetuses at different gestational ages; a database comprising developmental gene expression patterns established for karyotypically and developmentally normal male, and normal female fetuses at different gestational ages; and instructions for using the materials, databases and gene-expression array according to the methods of the invention.

The inventive kits may also contain materials to label samples of nucleic acids with a detectable agent. The detectable agent may comprise a fluorescent label, for example, a fluorescent dye, such as, Cy-3™, Cy 5™, Texas Red, FITC, phycoerythrin, rhodamine, fluorescein, fluorescein isothiocyanate, carbocyanine, merocyanine, styryl dye, oxonol dye, BODIPY dye, as well as equivalents, analogues, derivatives, and combinations of these compounds. Alternatively, the detectable agent may comprise a hapten, for example, a biotin/avidin system.

The inventive kits may also comprise hybridization and wash buffers, RNase inhibitor, carrier RNA and/or Human Cot-1 DNA.

The kits of the present invention optionally comprise different containers for each individual reagent. Each component will generally be suitable as aliquoted in its respective container. The container of the kits optionally includes at least one vial, ampoule, test tube, flask, or bottle. The individual containers of the kit are preferably maintained in close confinement for commercial sale.

### EXAMPLES

The following examples describe modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Most of the results presented in this section have been described by the Applicants in a recent scientific publication (P. B. Larrabbee et al., J. Am. Med. Assoc., February 2005, in print), which is incorporated herein by reference in its entirety.

### Materials and Methods

Amniotic Fluid Collection.

Approval was obtained from Tufts-New England Medical Center (Boston, Mass.) and Women and Infants' Hospital (Providence, R.I.) Institutional Review Boards to obtain amniotic fluid supernatant samples for the study reported herein.

Cases. In healthy pregnancies, between 10 and 30 mL of amniotic fluid can safely be removed from the fetal sac but only about 8 to 15 mL of supernatant remains following clinical testing, including karyotype analysis and alpha-fetoprotein measurement. Preliminary experiments showed that this remaining volume of amniotic fluid from a normal singleton fetus might not contain a sufficient quantity of mRNA for microarray analysis. Therefore, 5 large-volume amniotic fluid samples were obtained from 4 pregnant women undergoing therapeutic amnioreduction for polyhydramnios. Two of these women had fetuses with hydrops (gestational age of 29 4/7 weeks (Hydrops1) and 32 weeks (Hydrops2)), one had a fetus with twin-twin transfusion (TTT) syndrome (gestational age of 20 weeks (TTT2)) and another woman with fetal TTT underwent amnioreduction at two different gestational times (21 6/7 weeks (TTT3)and 24 3/7 weeks (TTT1)), and thus provided 2 samples. Cell-free supernatant was obtained by centrifugation of at least 350×g for 10 minutes.

Controls. In order to obtain sufficient RNA from healthy fetuses for comparison, multiple 10 mL samples of frozen, archived amniotic fluid supernatant were combined to form larger pools. These samples were obtained from pregnant women between 17 and 18 weeks of gestational age who underwent routine genetic amniocentesis for advanced maternal age. Six samples from male fetuses and 6 from female fetuses were selected based on known normal karyotypes and similar gestational age. These control samples were combined by gender, with each 60 mL pool representing amniotic fluid supernatant of an average 17-week fetus. Cell-free supernatant was obtained by centrifugation of 350×g for 10 minutes.

Oligonucleotide Hybridization

After centrifugation, total RNA was extracted from all samples using the QIAamp Viral RNA Vacuum Protocol for Large Sample Volumes (Qiagen, Inc., Valencia, Calif.) with modification. Modifications to this protocol included: (1) an increase in volume of buffer viral lysis buffer (AVL) and ethanol to 20 mL for each 5 mL of amniotic fluid per column and (2) the use of 60 mL syringes, which were attached to spin columns to accommodate the large sample volumes. Briefly, 5 mL of amniotic fluid supernatant was combined with 20 mL of Qiagen buffer AVL and mixed thoroughly. To this mixture 20 mL of 100% ethanol was added, and the sample was again mixed thoroughly. The solution was then applied to multiple QiaAmp columns by means of a vacuum manifold and syringe attachment. The bound nucleic acids were washed with Buffer AW1, then digested on the column with the Qiagen RNAse inhibitor-free DNase kit, followed by additional washes with buffers AW1 and AW2. RNA was eluted with 50 μL of RNAse inhibitor free water per column. The eluants were pooled and subjected to ethanol precipitation (2.5 volumes 100% EtOH, 3M NaAc, glycogen), except for a 3 μL aliquot that was diluted 12 fold and used for RT-PCR quantitation of the glyceraldehyde-3-phosphate dehydrogenase (GAPDH) transcript.

The mRNA was amplified twice and converted to cRNA by in vitro transcription in the presence of biotinylated nucleoside triphosphates as described in the GenChip® Eukaryotic Small Sample Target Labeling Technical Note (Affymetrix, Inc., Santa Clara, Calif.). This protocol is detailed at www.affymetrix.com. Briefly, total RNA was transcribed into cDNA using a T7-oligo dT primer, ensuring that only poly-A mRNA was targeted. A second strand of cDNA was then synthesized from the first. The double stranded cDNA was purified using a phase lock gel-phenol/chloroform extraction. From the double stranded cDNA an in vitro transcription was performed with the Ambion MEGAscript protocol. The in vitro transcripts were purified using RNAeasy columns (Qiagen) following the manufacturer's protocol. The cRNA was then subjected to another round of cDNA synthesis using random hexamers. The double stranded cDNA from this process was used for a second in vitro transcription reaction, this time with the Enzo BioArray transcript labeling kit, using biotinylated ribonucleotides. Samples were further purified by phenol-chloroform extraction using Phase Lock Gels (Eppendorf AG, Hamburg, Germany). To verify the quantity and quality of biotinylated cRNA, samples were analyzed using gel electrophoresis and fragmented before hybridization to Affymetrix Test3 oligonucleotide arrays according to the Affymetrix GeneChip® Expression Analysis Technical Manual (r3).

Subsequently, 15-75 μg of biotinylated cRNA were hybridized to Affymetrix HG-U133A arrays. As already mentioned above, these arrays are composed of 22,283 probe sets and over 500,000 distinct oligonucleotide features, representing 14,239 of the best characterized human genes (Affymetrix, Inc.).

Microarray Data Analysis and Statistical Analysis

Each array was scanned at 570 nm using a confocal scanner (Agilent, Palo Alto, Calif.) with a resolution of 3 μm/pixel. Pixel intensities were measured, and expression signals were extracted and analyzed using Microarray suite 5.0 (Affymetrix). All microarrays were scaled to the same target signal of 50 using the “All Probe Sets” scaling option, so that the expression signals from all experiments could be directly compared.

Comparison analyses were performed using the Wilcoxon signed rank test via the Microarray Suite 5.0 software between each of the TTT or hydrops cases and the pooled male control. Data from the TTT3 sample at 21 6/7 weeks and the pooled female sample were not used due to noisy data (see below). Data were copied into Excel field (Microsoft, v. 97 SR-2) and sorted from probe sets called “present” in either the case or control. Data for each case were then narrowed to transcripts that were increased or decreased relative to the pooled male control by a two-fold or greater difference. The two remaining TTT data sets were then compared to one another, as were the two hydrops data sets, to detect genes consistently increased or decreased in both cases with the same disease compared to the pooled control. Finally, expression levels of selected genes of interest, such as Y chromosome genes, surfactant, mucin, keratin, aquaporin, and placental genes were reviewed in all cases relative to the pooled male control.

### Example 1

### Preliminary Test—Fetal mRNA Extraction From Amniotic Fluid

Cell-free fetal mRNA has been successfully extracted and amplified from both fresh and frozen residual amniotic fluid samples. Amniotic fluid samples were initially collected for routine diagnostic purposes; the supernatant is usually discarded following karyotype analysis, while in therapeutic amniocentesis the entire sample is discarded. In the cytogenetics laboratory, samples were spun at 350×g for 10 minutes to remove cells for culture. Samples were centrifuged again at 13,000×g either upon receipt in the case of fresh samples, or immediately after thawing in the case of frozen samples. This ensured that the extracted RNA was truly extracellular.

RNA was extracted using the Qiagen Viral RNA mini kit following the vacuum protocol as described above. Sample starting volumes were typically 420 μL. Synthetic poly-A RNA (15-25 μg) was added to the sample during extraction as a carrier. RNA was concentrated into a final volume of 60 μL.

Initially mRNA was extracted from frozen samples, and was present at a concentration between 500 and 1000 pg/mL. To test whether RNA was degraded by the freeze/thaw process and/or the time lapse between drawing and freezing the sample, frozen samples were thawed and two 420 μL aliquots were drawn; one for immediate processing and one that was kept at 4° C. for three hours before being subjected to RNA extraction. In all cases, there was a significant loss of amplifiable RNA over the three-hour period. However, if the amniotic fluid was frozen immediately after acquisition, there was more RNA recovered from the frozen sample as compared to the fresh sample.

From these preliminary experiments, it appears that the extracellular RNA present in amniotic fluid at the time of sample acquisition degrades over time. However, there is also an increase in extracellular RNA that derives from lysis and degradation of amniocytes, either over time or from the freezing and thawing of a sample. To obtain the most accurate assessment of extracellular RNA, samples need to be cleared of all cells as soon as possible after being drawn. Samples then need to be processed immediately or subjected to the addition of RNAse inhibitor and frozen at −80° C.

### Example 2

### Large Volume Amniotic Fluid Samples—Processing and Storage

In some instances, large volumes (>1L) of amniotic fluid are drawn for therapeutic reasons (i.e., polyhydramnios). These samples, which are usually discarded, provide large starting quantities of fetal cell-free RNA. Nine of such high volume samples have been collected so far and are currently stored. Typically, the amniotic fluid was drawn into 1 L vacuum-sealed containers in a sterile manner. Upon receipt, the fluid was divided into 50 mL aliquots and centrifuged at 800×g for 15 minutes to remove any cellular material. The supernatant (45 mL) from the samples was pooled into 225 mL containers for storage at −80° C.

### Example 3

### Large Volume Amniotic Fluid Samples—RNA Extraction and Preparation for Microarrays

Two large volume amniotic fluid samples were obtained and processed as above. One was from the pregnant woman carrying twin female fetuses at 24 3/7 weeks of gestation (designated TTT1). The other sample was taken from the pregnant woman at 29 4/7 weeks of gestation whose male fetus had hydrops of unknown etiology (designated Hydrops1). In addition, 30 mL of each sample was taken (in 5 mL aliquots) and RNA was extracted in lieu of freezing the aliquot using a modification of the QIAamp Viral RNA Vacuum Protocol for Large Volumes (Qiagen, Inc., Valencia, Calif.) as described above. The two 30 mL samples yielded 7.2 ng and 12.7 ng, respectively. In order to obtain a larger quantity of RNA, 60 mL of previously frozen amniotic fluid supernatant from the above samples were used for an additional extraction. The samples were pooled, precipitated and resuspended in 10 μL of RNAse inhibitor free water, leading to final total yields of 17 ng (sample TTT1) and 36 ng (sample Hydrops1) (see Table 1).

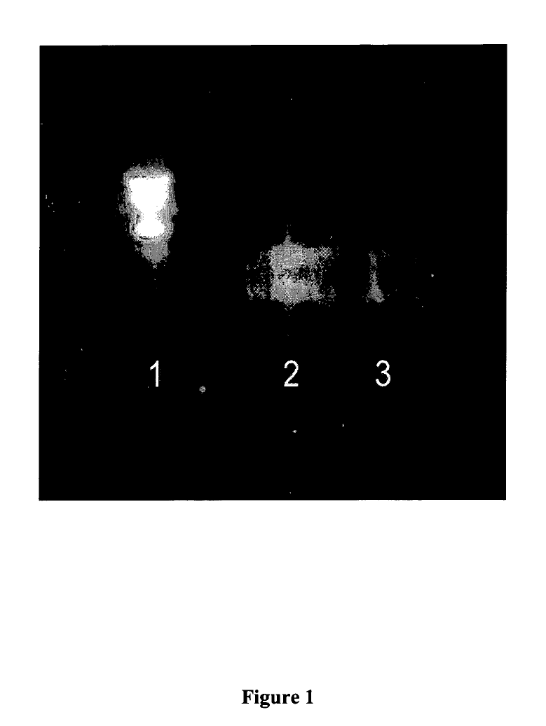

Samples were transcribed and labeled using methods from the GeneChip® Eukaryotic Small Sample Target Labeling Technical Note available from the Affymetrix website as described above. The labeled cRNA was subjected to chemical fragmentation in preparation for hybridization (see FIG. 1). Following amplification, UV spectrophotometer analysis demonstrated yields of 61.3 μg (sample TTT1) and 24.9 μg (sample Hydrops1) of labeled cRNA, which was more than adequate for hybridization to microarrays.

### Example 4

### Hybridization to Gene-Expression Microarrays

Initially, 5 μg of each sample was hybridized to a GeneChip® Test3 array, followed by antibody amplification of signals. The Test3 array contains a subset of 24 human genes that are expressed throughout development. Analysis of the data obtained suggested that cRNA was sufficient in quality and quantity for subsequent application to a microarray with a larger set of human genes.

The hybridization process was then repeated by loading 15 μg of labeled cRNA onto the Affymetrix gene expression microarray HG-U133A as described above. In the female samples, of the 22,283 probe sets on the microarray, 8,097 (36.3%) were present, 13,762 (61.8%) were absent, and 424 (1.9%) were marginally expressed. In the male sample, 9,864 (44.1%) were present, 11,992 (53.8%) were absent, and 457 (2.1%) were marginally expressed. The highest level of expression in both samples was found for many ribosomal protein transcripts, although there was no significant difference in the expression of the majority of these genes between the two samples. Increases or decreases in expression between two samples or groups of samples for each probe set were also determined by Wilcoxon's signed rank test (qualitative probability value) and Tukey's Biweight method (quantitative degree of change in base 2 logarithm).

The hybridization results in the two samples were then compared to each other. Of the 22,283 probe sets, there was no difference between the two samples in the level of expression of 18,266 (82%) of them. Importantly, a number of genes were expressed at significantly different levels between the two samples. This included 1,480 and 2,258 probe sets that were significantly decreased or increased, respectively, in the male amniotic fluid sample compared to the female sample. In addition, 121 and 158 probe sets were marginally decreased or increased, respectively, between the two samples.

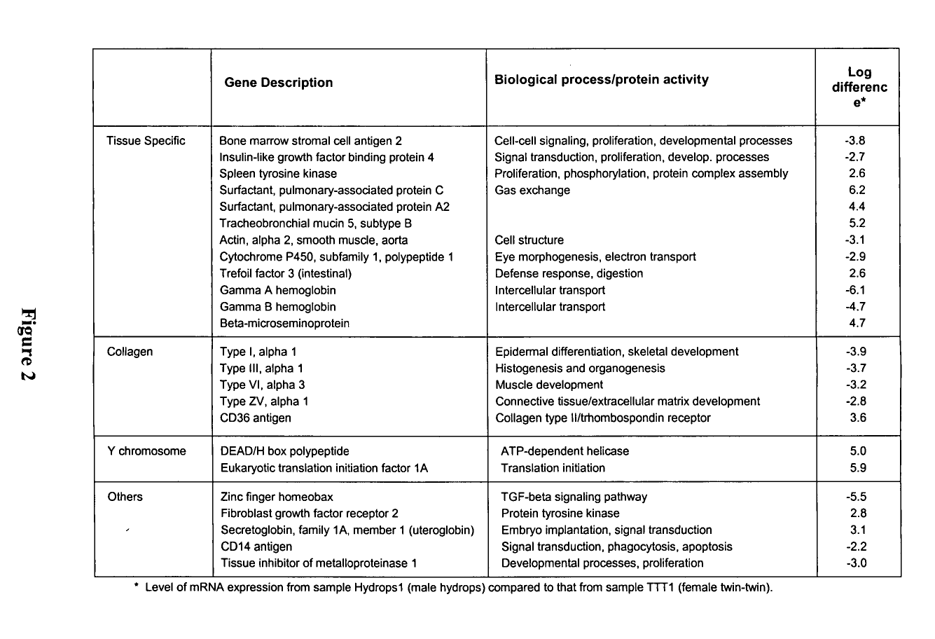

A subset of the genes that were expressed at different levels is shown in the table presented in FIG. 2. Since the amniotic fluid samples were from male and female fetuses, this allowed confirmation of the success of the microarray based on the presence or absence of Y chromosome-specific transcripts. Two of the genes found on the Y chromosome are shown in the table, both were expressed in the male sample and not in the female sample, as expected. As also shown in the table of FIG. 2, some of the genes that were expressed at different levels between the two samples represent those involved in differentiation and developmental processes. These differences in expression were also observed in many tissue specific genes as well as in families of genes (such as collagen). These preliminary results provide intriguing clues as to the underlying biology of TTT syndrome and hydrops. For future experiments, baseline levels of mRNA expression at different gestational ages will be established.

Overall, these data suggest that mRNA can be successfully extracted from amniotic fluid, amplified and hybridized to human gene expression microarrays. This implies that the naked mRNA in amniotic fluid is not totally degraded. Furthermore, the analytical method used is robust and the mining of large quantities of data is feasible. The results obtained showed definite differences in gene expression in the male and the female fetuses. The up-regulation of Y chromosome genes in the male fetus is very reassuring. On the other hand, the up-regulation of surfactant-associated genes in the fetus with hydrops is fascinating and deserves further inquiry. The preliminary data obtained show that this approach is very likely to lead to significant and new findings.

### Example 5

### Gene Expression Experiments and Analysis of Data

In order to obtain data from samples in addition to the two described in Example 3 and Example 4, three large samples of amniotic fluid were obtained from pregnant women undergoing therapeutic amnioreduction for polyhydramnios during either the second or third trimester. One woman had a fetus with hydrops, and two had fetuses with twin-twin transfusion (TTT) syndrome. One woman with fetal TTT, who previously provided sample TTT1, had undergone amnioreduction at two different gestational ages, and thus provided two samples.

Amniotic fluid samples from normal fetuses were also used for comparison purposes as described above.

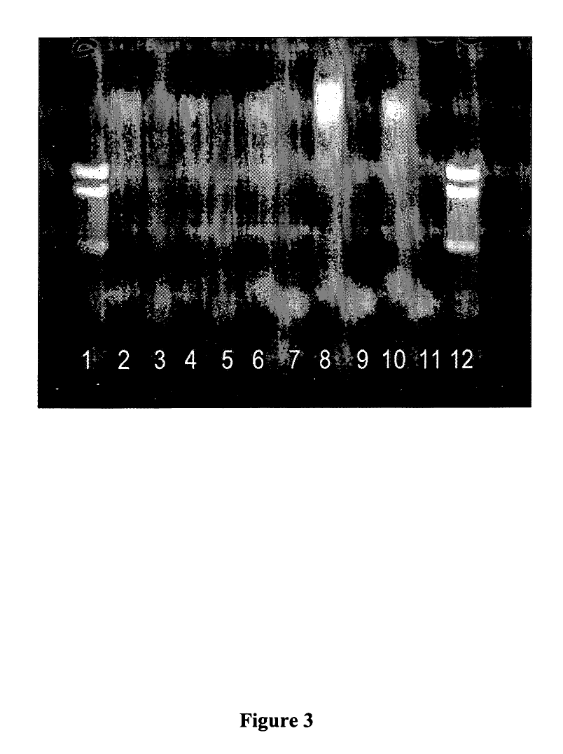

Amniotic fluid samples had been frozen at −80° C. Cells were removed prior to extraction or freezing by centrifugation at 800×g for 10 minutes at 4° C. RNA was extracted as described above . A total of 90 to 180 mL of amniotic fluid was extracted from the patients with TTT or hydrops and 57 to 62 mL for the two pooled samples from normal pregnancies (one male, one female). Next, the RNA was double-amplified, and converted to cRNA as described above. This yielded up to 8,000-fold amplification of extracted RNA, which suggests that smaller starting volumes of amniotic fluid material might be used. The samples were further purified by phenol-chloroform extraction and cRNA samples were fragmented (see FIG. 3), hybridized, stained and scanned as described above.

TABLE 1
Quantities of RNA extracted and amplified
from amniotic fluid samples.

| | Pooled | Pooled | | | | | |
|---|---|---|---|---|---|---|---|
| | male | female | | | | | |
| Fetal Sample | control | control | TTT2 | TTT3 | TTT1 | Hydrops1 | Hydrops2 |
| Gestational Age (weeks) | 17 | 17 | 20 | 21 6/7 | 24 3/7 | 29 4/7 | 32 |
| Volume Amniotic Fluid (mL) | 57 | 62 | 120 | 120 | 90 | 90 | 180 |
| Total RNA eluted (ng) | 46 | 77 | 105 | 22 | 17 | 36 | 20 |
| Biotinylated cRNA after | 59 | 36 | 84 | 54 | 61 | 25 | 65 |
| amplification (μg) | | | | | | | |

5 μg of biotinylated cRNAs were then hybridized to Affymetrix Test3 oligonucleotide arrays to determine the quality of labeled RNA. When the quality and quantity of the cRNA was determined to be sufficient, 15-75 μg of sample were then hybridized to Affymetrix U133A arrays.

Scanning of the arrays was carried out as described above and comparison analyses were performed using the Wilcoxon signed rank test via the Microarray Suite 5.0 software for each of the TTT or hydrops samples as the “cases” and the pooled normal karyotype male sample as the “control”.

Hybridization to Microarrays. Five of the samples tested hybridized well to the arrays (the two previously described in Example 3 and Example 4, as well as three additional samples: TTT2, Hydrops2, and the pooled male control), as measured by scale factors, which were within two-fold of one another, as recommended by the manufacturer. Two of the arrays (obtained with sample TTT3 and the pooled female control) were eliminated from analysis due to high scale factors and fewer transcripts called “present”, suggesting sample degradation. For the remaining five samples (two TTT samples, two hydrops, and the pooled male control), the overall average background (55.37 units, range 49.64-61.50) of the images was highly similar across all the arrays (typical values range from 20-100, per Affymetrix). Noise (Q), a measurement which reflects sample quality and electrical noise of the GeneArray™ Scanner, was also comparable across the arrays (median 2.21 units, range 2.06-2.37). Target values were set at 50 to minimize assay variability.

RNA Integrity. For the 5 analyzed samples, a median of 36% (range 11-44%) of the transcripts represented on the microarrays were detected as “present”, 62% (range 54-88%) were not detectable, i.e., “absent”, and 2% (range 1-2%) were “marginal”. There was evidence of low level of false- or cross-hybridization based on the presence of randomly distributed transcripts; these results were not statistically significant and were therefore not included for analysis. Three samples (Hydrops1, TTT1 and the pooled male control) hybridized very well to the arrays based on brightness of array signals, scale factors, and levels of some housekeeping gene transcripts, providing data that would be comparable to mRNA extracted from a tissue source. Two of these three samples contained at least a portion of mRNA that had been extracted immediately from fresh amniotic fluid. The pooled male control was made up entirely of frozen, archived amniotic fluid material.

Within individual samples, there was some variation in the 3′/5′ ratios of the internal control genes (GAPDH, and actin), that are used to assess RNA sample and assay quality. When these control genes were compared across all samples, certain control genes consistently had a normal (i.e., less than 3) 3′/5′ ratio in every sample while other control genes always had a high 3′/5′ ratio (10 to 100).

After hybridization was determined to work sufficiently well in five of the seven samples tested, the levels of gene transcripts included in the arrays were examined in order to explore whether the observed patterns would correlate with known variables between the cases and control: gender, gestational age and disease status.

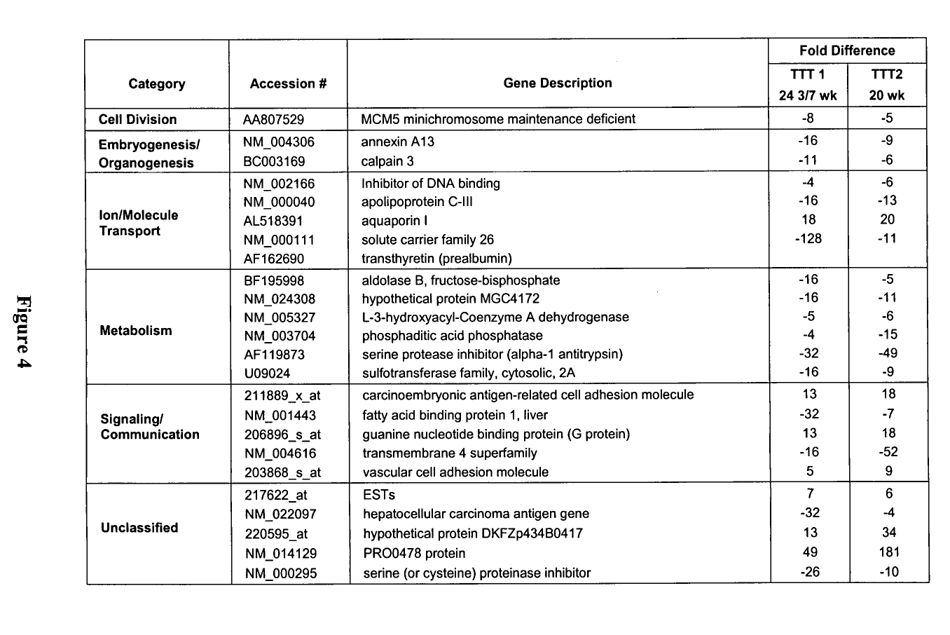

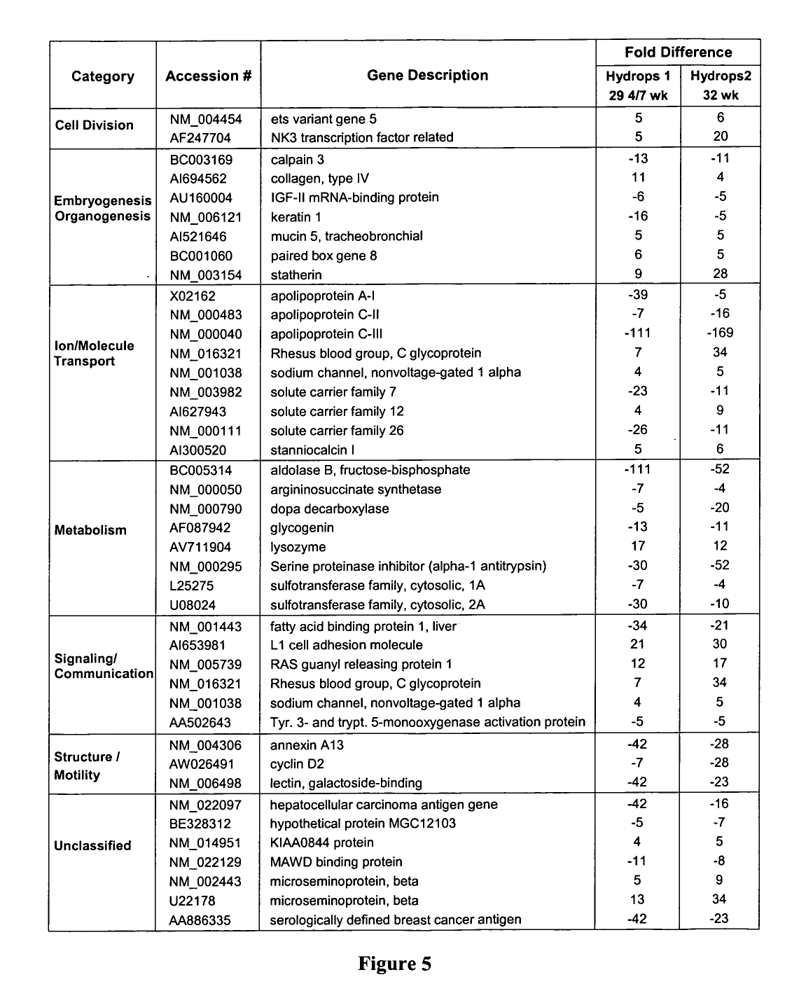

Differences in Gene Expression between Samples. Of the 22,283 transcripts present on the microarray, a median of 20% (range 15-29%) had significant differences in their levels of expression when comparing the cases and the pooled male control. The tables presented in FIG. 4 and FIG. 5 show a selection of genes with the most statistically significant different levels of expression (larger than 4 fold) in both TTT fetuses and both hydrops fetuses, respectively, compared to the pooled control.

Y chromosome genes: One Y-chromosome transcript (ribosomal protein S4, accession # NM_{—}001008) was expressed by all four samples from male fetuses (including the pooled control), but not by the sample from a female fetus. Additional Y chromosome transcripts were present in the two most successful male samples (the pooled male control and Hydrops1, see Table 2 below). The presence of Y-chromosome transcripts in all four male samples but not the female sample provided validation for the microarray data.

TABLE 2
Y chromosome genes, by patient and gestational age.

| | pooled | | | | | | | | | | | | |
|---|---|---|---|---|---|---|---|---|---|---|---|---|---|
| | control | TTT2 | | | TTT1 | | | Hydrops1 | | | Hydrops2 | | |
| | male | female | | | female | | | male | | | male | | |
| Title | 17 wk | 20 wk | | | 24 3/7 wk | | | 29 4/7 wk | | | 32 wk | | |
| (Map Location) | D | D | C | R | D | C | R | D | C | R | D | C | R |
| Ribosomal protein | + | + | ↓ | −2 | − | ↓ | −7 | + | NC | | + | ↓ | −1.6 |
| (Yp11.3) | | | | | | | | | | | | | |
| Translation initiation | | | | | | | | | | | | | |
| factor 1A(Yq11.221) | + | + | NC | | − | ↓ | −5 | + | NC | | − | NC | |
| Translation initiation | | | | | | | | | | | | | |
| factor 1A(Yq11.221) | + | + | NC | | − | NC | | + | ↑ | 1 | − | NC | |
| DEAD/H box | | | | | | | | | | | | | |
| Polypeptide(Yq11) | + | + | ↓ | −1 | − | ↓ | −4 | + | ↑ | 1 | − | NC | |
| DEAD/H box | | | | | | | | | | | | | |
| Polypeptide(Yq11) | + | + | NC | | − | ↓ | −2 | + | NC | | − | NC | |

D = Detection (+ = Present, − = Absent);
C = Chance (↑ = Increase, ↓ = Decrease, NC = No Change); and
R = Signal Log Ratio.

Data sets were screened for gene families related to fetal development to find changes in hybridization patterns with increasing gestational age. Intriguing differences were found in several genes expressed in lung, intestine and skin epithelial cells, which are all in contact with amniotic fluid. For example, Statherin (accession # NM_{—}003154), a gene involved in saliva secretion and ossification, was up to 28 times more concentrated in the fetuses of older gestational ages compared to the 17-week pooled control. Other examples of genes for which expression levels changes were consistent with fetal development include:

Surfactant: Surfactant genes (see Table 3), which are critical for fetal lung development, were found to increase with gestational age. The pooled 17-week control, the most immature fetal sample tested in this series of experiments, exhibited only three transcripts for surfactant protein B and only one of the five transcripts for surfactant protein C. By comparison, the Hydrops1 patient, at 29 4/7 weeks of gestation, expressed all surfactant genes: A, B, C and D (nine total transcripts). These findings were consistent with the published data. It is well-known that the type and quantity of surfactant genes expressed in human fetal lungs increase during development. mRNAs for surfactant proteins B and C are detectable as early as 13 weeks, and by 24 weeks, the levels are 50 and 15%, respectively of adult levels; surfactant protein A expression begins only after about 30 weeks and reaches maximum near term; and surfactant protein D mRNA is first detectable in the second trimester, with expression increasing throughout fetal and postnatal development (C. R. Mendelson, Ann. Rev. Physiol., 2000, 62: 875-915). The findings of the present study are consistent with the published data. All of the fetuses older than 24 weeks produced increased amounts of surfactant proteins B and C compared to the 17-week control, and surfactant proteins A and D were observed only after 29 weeks. The fact that the 20-week fetus and the 32-week fetus produced fewer surfactant transcripts than some of the more immature fetuses may be explained by their severe illnesses, or because these two microarrays did not hybridize as well as the others.

TABLE 3
Surfactant Pulmonary (SP) Associated Proteins, by patient and gestational age.

| | | Pooled | | | | | | | | | | | | |
|---|---|---|---|---|---|---|---|---|---|---|---|---|---|---|
| | | male | | | | | | | | | | | | |
| | | control | TTT2 | | | TTT1 | | | Hydrops1 | | | Hydrops2 | | |
| | | 17 wk | 20 wk | | | 24 3/7 wk | | | 29 4/7 wk | | | 32 wk | | |
| SP | Accession # | D | D | C | FD | D | C | FD | D | C | FD | D | C | FD |
| SP A2 | NM_006926 | − | − | | | − | | | + | ↑ | 20 | − | | |
| SP B | J02761 | + | | | | + | ↑ | 3 | + | ↑ | 4 | + | | |
| SP B | 4901244 | + | − | | | + | ↑ | 3 | + | ↑ | 5 | + | | |
| SP C | NM_003018 | − | − | | | − | | | + | ↑ | 64 | − | | |
| SP C | BC005913 | − | − | | | − | | | + | ↑ | 49 | − | | |
| SP C | AA633841 | − | − | | | − | | | + | ↑ | 49 | − | | |
| SP C | AI831055 | − | − | | | − | | | + | ↑ | 5 | − | | |
| SP C | 4878786_RC | + | − | | | + | ↑ | 3 | + | ↑ | 14 | + | ↑ | 6 |
| SP D | NM_003019 | − | − | | | − | | | + | ↑ | 3 | + | ↑ | 7 |

D = Detection (+ = Present, − = Absent);
C = Change (↑ = Increase); and
FD = Fold difference.

Mucin: Distinct patterns of expression in the mucin gene family were observed (see Table 4). There are 22 different transcripts representing 11 types of mucin present on the U133A array, and the majority of these transcripts were not present in any of the samples tested. However, the most mature fetuses expressed several important mucin genes. Mucins are filamentous glycoproteins present at the interface of epithelia and extracellular environments in the gastrointestinal tract, lungs, or urogenital tract (J. Dekker et al., Trends Biochem. Sci., 2002, 27: 126-131). As fetuses mature, they produce mucin in increasing amounts to protect their epithelia in preparation for life outside the womb. This idea is consistent with the increased expression of several mucins observed with advancing gestational age of the amniotic fluid samples. For example, tracheobronchial/gastric mucin 5, subtypes A and C, were found only in the two fetuses above 29 weeks. Salivary mucin 7 was found in four of the five fetuses, and in significantly increased concentrations in the older fetuses compared to the less mature ones. Expression levels of this transcript were found to increase with higher gestational age, and by 32 weeks gestational age, production was 64 times higher compared to the 17-week control.

TABLE 4
Mucin gene transcripts, by patient and gestational age*.

| | | Pooled | | | | | | | | | | | | |
|---|---|---|---|---|---|---|---|---|---|---|---|---|---|---|
| | | male | | | | | | | | | | | | |
| | | control | TTT2 | | | TTT1 | | | Hydrops1 | | | Hydrops2 | | |
| | | 17 wk | 20 wk | | | 24 3/7 wk | | | 29 4/7 wk | | | 32 wk | | |
| SP | Accession # | D | D | C | FD | D | C | FD | D | C | FD | D | C | FD |
| Mucin 1, transmembrane | NM_002456 | − | + | | | + | | | + | ↑ | 4 | + | | |
| Mucin 1, transmembrane | AI610869 | + | − | | | + | ↑ | 1 | + | ↑ | 4 | + | ↑ | 3 |
| Mucin 5, | AW192795 | − | − | | | − | | | + | ↑ | 20 | − | | |
| tracheobronchial/gastric | | | | | | | | | | | | | | |
| Mucin 5, | AI521646 | − | − | | | − | | | + | ↑ | 5 | + | ↑ | 5 |
| tracheobronchial/gastric | | | | | | | | | | | | | | |
| Mucin 7, salivary | L13283 | + | + | | | + | ↑ | 11 | + | ↑ | 20 | + | ↑ | 56 |

*A selection of transcripts with the most significant differences.
D = Detection (+ = Present, − = Absent);
C = Change (↑ = Increase); and
FD = Fold difference.

Keratin: The gene family keratin was found to be strongly expressed in all of the fetuses tested (see Table 5). Nineteen different types of keratin genes (28 transcripts) are represented on the U133A array. The control sample expressed all but one transcript for normal types of keratin, and did not express most of the transcripts for abnormal forms of keratin. The fetuses of more advanced gestational age also expressed most of the normal transcripts, but in significantly decreased amounts. In fact, the most mature fetuses, the 32-week Hydrops2, had a four-fold decrease in several of the keratin transcripts compared to the 17-week control.

TABLE 5
Keratin genes trasncripts, by patient and gestational age*.

| | | Pooled | | | | | | | | | | | | |
|---|---|---|---|---|---|---|---|---|---|---|---|---|---|---|
| | | male | | | | | | | | | | | | |
| | | control | TTT2 | | | TTT1 | | | Hydrops1 | | | Hydrops2 | | |
| | | 17 wk | 20 wk | | | 24 3/7 wk | | | 29 4/7 wk | | | 32 wk | | |
| Transcript | Accession # | D | D | C | FD | D | C | FD | D | C | FD | D | C | FD |
| keratin 4 | X07695 | + | + | ↓ | −3 | + | ↓ | −2 | + | | | + | | |
| keratin 5 | NM_000424 | + | − | | | + | ↓ | −1 | + | ↑ | 2 | + | | |
| keratin 6A | AL569511 | + | − | | | + | | | + | ↑ | 2 | + | | |
| keratin 6B | J00269 | + | − | | | + | ↓ | −2 | + | ↑ | 1 | + | | |
| keratin 6B | AI831452 | + | + | ↓ | −4 | + | | | + | | | + | | |
| keratin 7 | BC002700 | + | − | | | + | ↓ | −2 | + | | | + | | |
| keratin 8 | U76549 | + | + | ↓ | −3 | + | ↓ | −3 | + | ↓ | −2 | + | ↓ | −5 |
| keratin 10 | NM_000421 | + | − | | | + | ↓ | −3 | + | ↓ | −2 | + | ↓ | −2 |
| keratin 10 | M19156 | + | − | | | + | ↓ | −2 | + | ↓ | −2 | + | ↓ | −3 |
| keratin 10 | X14487 | + | + | ↓ | −2 | + | | | + | | | + | ↓ | −3 |
| keratin 13 | NM_002274 | + | + | ↓ | −6 | + | | | + | | | + | | |
| keratin 14 | BC002690 | + | + | | | + | | | + | ↑ | 3 | + | ↑ | 2 |
| keratin 15 | NM_002275 | + | − | | | + | | | + | ↑ | 2 | + | | |
| keratin 16 | AF061812 | + | + | ↓ | −6 | + | ↓ | −2 | + | ↑ | 1 | + | | |
| keratin 17 | NM_000422 | + | + | ↓ | −3 | + | ↓ | −3 | + | ↓ | −2 | + | ↓ | −5 |
| keratin 17 | Z19574 | + | + | | | + | ↓ | −3 | + | | | + | | |
| keratin 18 | NM_000224 | + | + | ↓ | −3 | + | ↓ | −3 | + | ↓ | −2 | + | ↓ | −3 |
| keratin 19 | NM_002276 | + | + | | | + | | | + | | | + | ↓ | −2 |
| keratin 23 | NM_015515 | + | − | | | − | | | + | ↓ | −5 | + | | |
| keratin 24 | NM_019016 | + | + | | | + | ↓ | −17 | + | ↑ | 1 | + | | |

*A selection of transcripts with the most significant differences.
D = Detection (+ = Present, − = Absent);
C = Change (↑ = Increase, ↓ = Decrease); and
FD = Fold difference.

Keratins are produced by the kidney, intestine and skin. In the skin, keratin proteins are first made in the intermediate layer during the 11^{th} week of human fetal development. During the fifth month, this layer develops into definitive layers of keratinocytes, and as cells progress from the basal layer of stem cells to the outer horny layer, they stop producing keratins, which are then bundled and cross-linked. When reaching the top layer, metabolic activity of the cells has ceased, with the scalelike terminally differentiated keratinocytes forming the horny protective layer (“Human embryology,” W. J. Larsen, 1993 (1^{st} Ed.), Churchill Livingstone: New York, N.Y.)

The reason for the observed gestational age-related decrease in keratin expression may be that as the fetal skin matures, fewer keratin-producing cells are in direct contact with the amniotic fluid, and may not release their mRNA into the cell-free fraction. Rather, the layer of hard, cross-linked keratin itself may protect the buried keratin-producing cells from releasing their mRNA into amniotic fluid.

Other genes were reviewed in the context of fetal pathology or maternal-placental-fetal trafficking of cell-free nucleic acids. These include:

Aquaporin: Aquaporin genes are water transporters, and, as such, may be expected to play a role in polyhydramnios. Indeed, a 16-fold elevation in aquaporin 1 was observed in transcripts from the two TTT fetuses compared to the control (see Table 6). Aquaporin 1 has been shown in previous studies to be expressed in fetal membranes. The hydrops fetuses did not show this same increase. In the fetuses tested here, low levels of expression of only one of the three transcripts for aquaporin 3, a gene which was not found in fetal membranes in the same study as aquaporin 1. There was minimal difference in expression of aquaporin 3 between any of the cases (TTT or hydrops) compared to the control.

TABLE 6
Aquaporin genes, by patient and gestational age*.

| | | Pooled | | | | | | | | | | | | |
|---|---|---|---|---|---|---|---|---|---|---|---|---|---|---|
| | | male | | | | | | | | | | | | |
| | | control | TTT2 | | | TTT1 | | | Hydrops1 | | | Hydrops2 | | |
| | | 17 wk | 20 wk | | | 24 3/7 wk | | | 29 4/7 wk | | | 32 wk | | |
| Transcript | Accession # | D | D | C | FD | D | C | FD | D | C | FD | D | C | FD |
| aquaporin 1 | NM_000385 | − | + | | | + | | | − | | | + | ↑ | 3 |
| aquaporin 1 | AL518391 | − | + | ↑ | 20 | + | ↑ | 18 | − | | | − | | |
| aquaporin 3 | NM_004925 | − | − | | | − | | | − | | | − | | |
| aquaporin 3 | 4855867_RC | + | − | | | + | ↓ | −2 | + | | | + | | |
| aquaporin 3 | 4855868 | − | − | | | − | | | − | | | − | | |

*Fetuses with TTT or hydrops relative to the pooled male control.
D = Detection (+ = Present, − = Absent);
C = Change (↑ = Increase, ↓ = Decrease); and
FD = fold difference.

There is some evidence that aquaporin 1 is present on the apical aspect of the chorionic plate amnion but aquaporin 3 is not active in the fetal membranes. It has been postulated that aquaporin 1 may play a role in water movement from the amniotic cavity across the placenta into the fetal circulation. (S. E. Mann et al., Am. J. Obstet. Gynecol., 2002, 187: 902-907). Our findings support the presence of aquaporin 1 and the relative lack of aquaporin 3 in amniotic fluid. The significant increase of aquaporin 1 in TTT patients suggests that it might play a role in the polyhydranmios associated with TTT but not hydrops.

Placenta Genes: Genes specific for the placenta, including corticotropin releasing hormone, chorionic somatomammotropin hormone 1 (placental lactogen), and the beta subunit of human chorionic gonadotropin were examined in the amniotic fluid because their presence in the plasma of pregnant women provides proof of fetal-maternal trafficking of cell-free RNA (E. K. Ng et al., Proc. Nat. Acad. Sci., 2003, 100: 4748-4753 and E. K. Ng et al., Clin. Chem., 2003, 49: 727-731). Transcripts for these genes (8 total) were not expressed in any of the samples tested. The absence of placenta-specific genes in the amniotic fluid supports the idea that fetal-maternal trans-placental transfer of nucleic acids is primarily one way: toward the mother. Previous work has shown that cell-free fetal DNA in maternal plasma is significantly more concentrated relative to cell-free maternal DNA in the fetal plasma. (A. Sekizawa et al., Hum. Gen., 2003; 113: 307-310).

### Discussion

This is, to the best of the Applicant's knowledge, the first in vivo study of global gene expression in the living human fetus by oligonucleotide microarray analysis of fetal mRNA isolate from cell-free amniotic fluid. Cell-free fetal RNA was successfully extracted from this typically discarded component of amniotic fluid, amplified, labeled, and hybridized to oligonucleotide microarrays.

The analyses performed in this study revealed important information about the presence and level of gene expression in living human fetuses. In addition, this preliminary data appears to show that observed gene expression patterns correlated with known variables (gender, gestational age and disease status) between the cases and control. While it would have been optimal to validate the results obtained using real-time quantitative reverse transcriptase PCR, this was not possible due to limited sample template. However, the presence of one Y chromosome transcript in all 4 male samples but not the female sample provided physiologic validation of the data. Expression differences were then evaluated in gene families known to be important in fetal development to look for changes with gestational age. Significant differences were observed in several genes expressed in lung, intestine, and skin epithelial cells, which are all in contact with the amniotic fluid.

This study demonstrated that for the majority of samples, cell-free RNA from amniotic fluid successfully hybridized to microarrays. However, some samples hybridized less well, possibly because the RNA was degraded, which could occur from delays prior to sample processing or introduction of a freeze/thaw cycle. However, freezing and thawing did not appear to be detrimental to the male control, which hybridized well despite its composition of archived amniotic fluid samples that had been stored at −80° C. Additionally, it has been demonstrated that a single freeze/thaw cycle produces no significant effect on the cell-free RNA concentration in plasma or serum (N. B. Tsui et al., Clin. Chem., 2002, 48: 1647-1653). It is possible that cell-free RNA is inherently degraded, and therefore has different properties than RNA extracted from whole cells. RNA is labile, so it is surprising that any cell-free RNA in amniotic fluid survives until extraction. There is evidence that circulating RNA in plasma is associated with stabilizing particles (E. K. Ng et al., Clin. Chem., 2002, 48: 1212-1217). In this study, certain internal control genes had normal 3′/5′ ratios in every sample, while ratios of certain other genes were always high. This discrepancy suggests a pattern of preservation of specific RNA transcripts, which could be related to alteration and packaging of mRNA during apoptosis. Housekeeping genes vary significantly in their expression patterns between various tissues and organisms (L. L. Hsiao et al., Physiol. Genomics, 2001, 7: 97-104), and these patterns in cell-free RNA in amniotic fluid are unknown. In addition, the kinetics of cell-free RNA in amniotic fluid have yet to be explored.

The low levels of non-significant false positives observed in this study could be due to cross-hybridization of the short oligonucleotide probes on the arrays with different mRNAs that have short sequences in common. However, the Affymetrix algorithms take this into account and have largely eliminated this source of error (J. Li et al., Toxicol. Sci., 2002, 69: 383-390). Additionally, the cases and control had different genetic backgrounds and other variables (gestational age and disease status) because amniocentesis is not generally performed on healthy fetuses greater than 19-20 weeks. Further, small amounts of maternal contamination could possibly confound the results. Therefore, the data on diversity of fetal gene expression must be interpreted with caution at this stage of investigation and further study is necessary before firm conclusions can be drawn.

For this pilot study, large volume samples were used to demonstrate feasibility. However, is appears that amniotic fluid samples from healthy fetuses contain a higher concentration of cell-free mRNA than amniotic fluid samples from fetuses with polyhydramnios (see Table 1). Technical improvements are directed toward improving extraction of mRNA from routinely collected amniotic fluid samples (<30 mL) so that in the future, individual fetuses may be studied.

In summary, this study demonstrates that cell-free feral mRNA can be isolated from amniotic fluid and successfully detected using oligonucleotide microarrays. Preliminary gene expression analyses appear to show gene expression patterns that vary among fetuses of different genders, gestational ages, and disease state. The entire study was conducted using a portion of amniotic fluid that is typically discarded, and thus is readily available for use in future studies. The intriguing gene expression differences observed suggest that this technology could facilitate the advancement of human developmental research as well as the cultivation of new biomarkers for assessment of the living fetus.

Future studies include comparison of gene expression profiles from amniotic fluid in healthy and abnormal fetuses to show whether certain genes are up regulated or down regulated. In this way, novel patterns of gene expression may be detected in abnormal fetuses (for example aneuploid fetuses), which could potentially serve as future targets for amplification in maternal blood. Amniotic fluid from fetuses undergoing lung maturity testing will also be examined to determine if a profile of normal or delayed pulmonary gene expression can be established. Since amniotic fluid is obtained for a variety of different clinical indications, the present approach may constitute a new way of assessing fetuses with growth or developmental problems while in utero.

In addition, preliminary experiments reported in Example 5 have provided interesting results revealing variations in the expression of different genes with gestational age. These variations, such as, for example, those observed for surfactant genes and genes of the mucin family, will be further investigated, with the ultimate goal of establishing a normal gene expression profile for healthy fetuses in each gestational week so that fetuses with problems can be compared.

What is claimed is:

1. Isolated amniotic fluid fetal RNA.

2. The fetal RNA of claim 1 obtained by a process comprising: treating a sample of amniotic fluid obtained from a pregnant woman such that fetal RNA present in the amniotic fluid is extracted, resulting in amniotic fluid fetal RNA.

3. The fetal RNA of claim 2, wherein cell populations are removed from the sample of amniotic fluid prior to treating the sample.

4. The fetal RNA of claim 2, wherein treating the sample of amniotic fluid comprises:
isolating cell populations present in the amniotic fluid;
optionally culturing the isolated cells; and
extracting fetal RNA from the isolated cells.

5. The fetal RNA of claim 1, wherein the amniotic fluid fetal RNA is fetal messenger RNA (mRNA).

6. A method of prenatal diagnosis comprising steps of:
providing a sample of amniotic fluid fetal RNA;
analyzing the amniotic fluid fetal RNA to obtain information regarding fetal RNA; and
based on the information obtained, providing a prenatal diagnosis.

7. The method of claim 6, wherein the amniotic fluid fetal RNA is obtained by treating a sample of amniotic fluid obtained from a pregnant woman such that fetal RNA present in the amniotic fluid is extracted and made available for analysis, resulting in amniotic fluid fetal RNA.

8. The method of claim 7, further comprising: removing cell populations from the sample of amniotic fluid prior to the treating step, resulting in a remaining amniotic material.

9. The method of claim 6, wherein the amniotic fluid fetal RNA is obtained by:
removing cell populations from a sample of amniotic fluid obtained from a pregnant woman, resulting in isolated cells;
optionally culturing the isolated cells; and
treating the isolated cells such that fetal RNA present in the cells is extracted and made available for analysis, resulting in amniotic fluid fetal RNA.

10. The method of claim 6, further comprising, prior to the analyzing step: (a) amplifying the amniotic fluid fetal RNA, resulting in amplified fetal RNA and optionally labeling the amplified fetal RNA with a detectable agent, (b) fragmenting the amniotic fluid fetal RNA, resulting in fragmented fetal RNA and optionally labeling the fragmented fetal RNA with a detectable agent, (c) converting the amniotic fluid fetal RNA into complementary DNA (cDNA), resulting in fetal cDNA and optionally labeling the fetal cDNA with a detectable agent, or (d) converting the amniotic fluid fetal RNA into complementary RNA (cRNA), resulting in fetal cRNA and optionally labeling the fetal cRNA with a detectable agent.

11. The method of claim 10, wherein the detectable agent is selected from the group consisting of a fluorescent label, a calorimetric label, a chemiluminescent label, a radionuclide, a magnetic label, a hapten, a microparticle, an enzyme, a detectable biological molecule and any combination thereof.

12. The method of claim 10, wherein analyzing the amniotic fluid fetal RNA comprises one or more of: determining the quantity of amniotic fluid fetal RNA, determining the concentration of amniotic fluid fetal RNA, determining the sequence composition of amniotic fluid fetal RNA, submitting the amniotic fluid fetal RNA to a gene analysis, and analyzing the amniotic fluid fetal RNA using an array.

13. The method of claim 6, wherein the information obtained from analyzing the amniotic fluid fetal RNA is selected from the group consisting of quantity of fetal RNA, concentration of fetal RNA, sequence composition of fetal RNA, qualitative fetal gene expression, quantitative fetal gene expression, and any combination thereof.

14. The method of claim 6, wherein providing a prenatal diagnosis comprises one or more of: determining fetal gender, determining fetal developmental progress, and identifying a disease or condition affecting the fetus.

15. The method of claim 6, wherein the prenatal diagnosis is performed for a fetus suspected of having a disease or condition.

16. A method for establishing gene expression in a fetus, the method comprising steps of:
providing a test sample of amniotic fluid fetal RNA, wherein the fetal RNA comes from a sample of amniotic fluid obtained from a pregnant woman, and wherein the test sample comprises a plurality of nucleic acid segments labeled with a detectable agent;
providing a gene-expression array comprising a plurality of genetic probes, wherein each genetic probe is immobilized to a discrete spot on a substrate surface to form the array;
contacting the array with the test sample under conditions wherein the nucleic acid segments in the sample specifically hybridize to the genetic probes on the array;
determining the binding of individual nucleic acid segments of the test sample to individual genetic probes immobilized on the array to obtain a binding pattern; and
based on the binding pattern obtained, establishing a gene expression pattern for the fetus.

17. The method of claim 16 further comprising: correlating one or more feature(s) of the gene expression pattern with fetal gender or correlating one or more feature(s) of the gene expression pattern with gestational age.

18. The method of claim 16, wherein the fetus is karyotypically and developmentally normal, karyptypically abnormal, developmentally abnormal, or affected with a clinical condition.

19. The method of claim 16 further comprising:
repeating all the previous steps for a statistically significant number of amniotic fluid fetal RNA samples from karyotypically and developmentally normal male or female fetuses of different gestational ages; and
based on the gene expression patterns obtained, establishing baseline levels of mRNA expression at different gestational ages in karyotypically and developmentally normal male or female fetuses.

20. The method of claim 16 further comprising: correlating one or more feature(s) of the gene expression pattern with a time or event in fetal development of a karyotypically and developmentally normal male fetus if the amniotic fluid fetal RNA analyzed comes from a male fetus, or with a time or event in fetal development of a karyotypically and developmentally normal female fetus if the amniotic fluid fetal RNA analyzed comes from a female fetus.

21. The method of claim 16 further comprising steps of:
repeating all the previous steps for a statistically significant number of amniotic fluid fetal RNA samples from karyotypically and developmentally normal male or female fetuses of different gestational ages;
correlating one or more feature(s) of the gene expression patterns obtained with a time or event in fetal development of a karyotypically and developmentally normal male or female fetus; and
based on the correlations, establishing a developmental gene expression pattern for karyotypically and developmentally normal male or female fetuses at different gestational ages.

22. The method of claim 16 further comprising steps of:
repeating all the previous steps for a statistically significant number of amniotic fluid fetal RNA samples from karyotypically abnormal fetuses with an identical chromosomal abnormality;
comparing each gene expression pattern obtained with baseline levels of mRNA expression established for karyotypically and developmentally normal fetuses of similar gestational age and gender;
based on the comparison, identifying one or more gene(s) abnormally expressed in the karyotypically abnormal fetuses, and associated with the chromosomal abnormality;
and optionally cataloguing the one or more gene(s) identified as a function of chromosomal abnormality

23. The method of claim 16 further comprising steps of:
repeating all the previous steps for a statistically significant number of amniotic fluid fetal RNA samples from developmentally abnormal fetuses with an identical developmental disease or condition;
comparing each gene expression pattern obtained with baseline levels of mRNA expression established for karyotypically and developmentally normal fetuses of similar gestational age and gender;
based on the comparison, identifying one or more gene(s) abnormally expressed in the developmentally abnormal fetuses, and associated with the developmental disease or condition; and
optionally cataloguing the one or more gene(s) identified as a function of developmental disease or condition.

24. The method of claim 16 further comprising steps of:
repeating all the previous steps for a statistically significant number of amniotic fluid fetal RNA samples from diseased fetuses affected with an identical clinical condition;
comparing each gene expression pattern obtained with baseline levels of mRNA expression established for karyotypically and developmentally normal fetuses of similar gestational age and gender;
based on the comparison, identifying one or more gene(s) abnormally expressed in the diseased fetuses, and associated with the clinical condition; and optionally cataloguing the one or more gene(s) identified as a function of clinical condition.

25. The method of claim 16, wherein the amniotic fluid fetal RNA is obtained by:
treating a sample of amniotic fluid obtained from a pregnant woman such that fetal RNA present in the amniotic fluid is extracted and made available for analysis, resulting in amniotic fluid fetal RNA.

26. The method of claim 25, further comprising: removing cell populations from the sample of amniotic fluid prior to the treating step, resulting in a remaining amniotic material.

26. The method of claim 16, wherein the amniotic fluid fetal RNA is obtained by:
removing cell populations from a sample of amniotic fluid obtained from a pregnant woman, resulting in isolated cells;
optionally culturing the isolated cells; and
treating the isolated cells such that fetal RNA present in the cells is extracted and made available for analysis.

27. The method of claim 16, further comprising, prior to the contacting step: (a) amplifying the amniotic fluid fetal RNA, resulting in amplified fetal RNA and optionally labeling the amplified fetal RNA with a detectable agent, (b) fragmenting the amniotic fluid fetal RNA, resulting in fragmented fetal RNA and optionally labeling the fragmented fetal RNA with a detectable agent, (c) converting the amniotic fluid fetal RNA into complementary DNA (cDNA), resulting in fetal cDNA and optionally labeling the fetal cDNA with a detectable agent, or (d) converting the amniotic fluid fetal RNA into complementary RNA (cRNA), resulting in fetal cRNA and optionally labeling the fetal cRNA with a detectable agent.

28. The method of claim 27, wherein the detectable agent is selected from the group consisting of a fluorescent label, a colorimetric label, a chemiluminescent label, a radionuclide, a magnetic label, a hapten, a microparticle, an enzyme, a detectable biological molecule and any combination thereof.

29. The method of claim 16, wherein determining the binding of individual nucleic acid segments of the test sample to individual genetic probes immobilized on the array to obtain a binding pattern comprises: measuring the intensity of the signals produced by the detectable agent at each discrete spot on the array.

30. The method of claim 16, wherein determining the binding of individual nucleic acid segments of the test sample to individual genetic probes immobilized on the array to obtain a binding pattern comprises steps of:
using a computer-assisted imaging system to obtain a fluorescence image of the array after hybridization; and
using a computer-assisted image analysis system to analyze the fluorescence image obtained, to interpret data imaged from the array and to display results as fluorescence intensity as a function of genomic locus.

31. A method of prenatal diagnosis performed by submitting amniotic fluid fetal RNA to an array-based gene-expression analysis, the method comprising steps of:
providing a test sample of amniotic fluid fetal RNA, wherein the fetal RNA comes from a sample of amniotic fluid obtained from a woman pregnant with a fetus of known gender and gestational age, and wherein the test sample comprises a plurality of nucleic acid segments labeled with a detectable agent;
providing a gene-expression array comprising a plurality of genetic probes, wherein each genetic probe is immobilized to a discrete spot on a substrate surface to form the array;
contacting the array with the test sample under conditions wherein the nucleic acid segments in the sample specifically hybridize to the genetic probes on the array;
determining the binding of individual nucleic acid segments of the test sample to individual genetic probes immobilized on the array to obtain a binding pattern;
based on the binding pattern obtained, establishing a gene expression pattern for the fetus;
analyzing the gene expression pattern; and
based on the analysis, providing a prenatal diagnosis.

32. The method of claim 31, wherein analyzing the gene expression pattern comprises:
comparing the gene expression pattern of the fetus to baseline levels of mRNA expression established for karyotypically and developmentally normal fetuses of identical gender and gestational age or to a developmental gene expression pattern established for karyotypically and developmentally normal fetuses of identical gender and gestational age.

33. The method of claim 31, wherein analyzing the gene expression pattern comprises:
detecting one or more gene(s) abnormally expressed.

34. The method of claim 33, wherein the one or more gene(s) abnormally expressed is/are associated with a chromosomal abnormally, a developmental anomaly and/or a clinical condition.

35. The method of claim 31, wherein providing a prenatal diagnosis comprises determining developmental progress of the fetus and/or identifying a disease or condition affecting the fetus.

36. The method of claim 31, wherein the fetus is suspected of having a disease or condition selected from the group consisting of a disease or condition associated with a chromosomal abnormality, a disease or condition associated with a developmental anomaly, a clinical condition and any combination thereof.

37. The method of claim 31, wherein the amniotic fluid fetal RNA s obtained by:
treating a sample of amniotic fluid obtained from a pregnant woman such that fetal RNA present in the amniotic fluid is extracted and made available for analysis, resulting in amniotic fluid fetal RNA.

38. The method of claim 37, further comprising: removing cell populations from the sample of amniotic fluid prior to the treating step, resulting in a remaining amniotic material.

39. The method of claim 31, wherein the amniotic fluid fetal RNA is obtained by:
removing cell populations from a sample of amniotic fluid obtained from a pregnant woman, resulting in isolated cells;
optionally culturing the isolated cells; and
treating the isolated cells such that fetal RNA present in the cells is extracted and made available for analysis.

40. The method of claim 31, further comprising, prior to the analyzing step: (a) amplifying the amniotic fluid fetal RNA, resulting in amplified fetal RNA and optionally labeling the amplified fetal RNA with a detectable agent, (b) fragmenting the amniotic fluid fetal RNA, resulting in fragmented fetal RNA and optionally labeling the fragmented fetal RNA with a detectable agent, (c) converting the amniotic fluid fetal RNA into complementary DNA (cDNA), resulting in fetal cDNA and optionally labeling the fetal cDNA with a detectable agent, or (d) converting the amniotic fluid fetal RNA into complementary RNA (cRNA), resulting in fetal cRNA and optionally labeling the fetal cRNA with a detectable agent.

41. The method of claim 40, wherein the detectable agent is selected from the group consisting of a fluorescent label, a calorimetric label, a chemiluminescent label, a radionuclide, a magnetic label, a hapten, a microparticle, an enzyme, a detectable biological molecule and any combination thereof.

42. The method of claim 31, wherein determining the binding of individual nucleic acid segments of the test sample to individual genetic probes immobilized on the array to obtain a binding pattern comprises steps of:
using a computer-assisted imaging system to obtain a fluorescence image of the array after hybridization; and
using a computer-assisted image analysis system to analyze the fluorescence image obtained, to interpret data imaged from the array and to display results as fluorescence intensity as a function of genomic locus.

43. A kit comprising the following components:
materials to extract cell-free fetal RNA from a sample of amniotic fluid obtained from a pregnant woman;
a gene expression array comprising a plurality of genetic probes, wherein each genetic probe is immobilized to a discrete spot on a substrate surface to form the array;
a database comprising baseline levels of mRNA expression established for karyotypically and developmentally normal male, and normal female fetuses at different gestational ages;
a database comprising developmental gene expression patterns established for karyotypically and developmentally normal male, and normal female fetuses at different gestational ages; and
instructions for using the materials, and array as set forth in claim 31.

44. The kit of claim 43 further comprising one or more components of the group consisting of materials to label a sample of nucleic acid with a detectable agent, hybridization buffer, wash buffer, RNase inhibitor, carrier RNA, and Human Cot-1 DNA.
